(19) United States
(12) Patent Application Publication
(10) Pub. No.: US 2026/0317375 A1
(43) Pub. Date: Oct. 15, 2026

(54) STEREOCOMPLEXED SELF-ASSEMBLING PEPTIDE HYDROGEL GENERATES SPIRAL NANOMORPHOLOGIES

(71) Applicant: UNIVERSITY OF VIRGINIA PATENT FOUNDATION, Charlottesvile, VA (US)

(72) Inventors: Kyle Jay Lampe, Charlottesville, VA (US); Paul W. Eisold, Pipersville, PA (US); Jonathan Florian, Washington, PA (US)

(21) Appl. No.: 19/643,457

(22) Filed: Apr. 9, 2026

Related U.S. Application Data
(60) Provisional application No. 63/786,112, filed on Apr. 9, 2025.

(51) Int. Cl. C07K 7/06; A61K 9/06; A61K 38/00; C12N 5/00

(52) U.S. Cl. CPC C07K 7/06; A61K 9/06; C12N 5/0068; A61K 38/00

(57) ABSTRACT

This disclosure relates to novel self-assembling pentapeptides and peptides containing such self-assembling pentapeptides, self-assembled hydrogels, and methods of making and using the same are described. These pentapeptides, and peptides containing such pentapeptides, can self-assemble under physiological conditions into spiral nanostructures. These hydrogels are useful in various applications, including but not limited to, scaffolds for tissue engineering, 2-dimensional (2-D) and 3-dimensional (3-D) cell cultures, drug delivery and encapsulation of therapeutic agents (cells, molecules, drugs, compounds), injectables (including those that gel in situ, such as hemostatic compositions), hemostatic agents, wound dressings, pharmaceutical carriers or vehicles, cell transplantation, cell storage, virus culture, and virus storage.

### CROSS-REFERENCE TO RELATED APPLICATIONS

This application claims priority to U.S. Provisional Application No. 63/786,112, filed Apr. 9, 2025. The contents of which are herein incorporated by reference in their entirety.

### STATEMENT REGARDING FEDERALLY SPONSORED RESEARCH OR DEVELOPMENT

This invention was made with government support under grant number 2104723, awarded by the National Science Foundation. The government has certain rights in the invention.

### REFERENCE TO AN ELECTRONIC SEQUENCE LISTING

A Sequence Listing accompanies this application and is submitted as an XML file of the sequence listing named “18250600087_SL.xml” which is 8,772 bytes in size and was created on Apr. 7, 2026. The sequence listing is electronically submitted via Patent Center with the application and is incorporated herein by reference in its entirety.

### BACKGROUND

An attractive approach to tissue regeneration relies on biomaterials that can mimic features of the native extracellular matrix (ECM), including proteolytic remodeling, cell-adhesion, and mechanical properties, while providing suitable induction for specific differentiation. Tissue engineering using biologically relevant hydrogel culture systems may improve regeneration as they have a broad range of structural flexibility, biological activity, and similar mechanics to native tissue, leading to more physiologically relevant cell behavior. However, an appropriate microenvironment that retains relevant biological and structural functions remains to be developed.

Hydrogels are a promising class of biomaterials composed of water-swollen polymeric networks that mimic several biological and mechanical properties of the naturally compliant human brain tissue. Despite the many technological advances in recreating the cellular and matrix microenvironments found in vivo, few materials exist that capture the highly dynamical and adaptable nature of the native ECM. Furthermore, even fewer materials have been designed that 1) are simple and inexpensive to synthesize, 2) gel in response to cytocompatible stimuli such as small shifts in pH or temperature, and 3) support and regulate cell function as a substitute for their normal physiological microenvironment. Transplanting cells, such as stem cells, may improve behavioral recovery following an injury or insult, or during chronic or degenerative diseases. Further, recent developments in neural tissue regeneration applications require direct injection of cells to the stroke region. However, transplant cell viability is known to be poor, and this is at least in part due to negative effects of damage done during the process of injection where cells undergo stresses such as non-physiological elongational flow and superphysiological shear forces. And thus, fabricating injectable and self-healing hydrogels to serve as cell carriers for transplantation therapies will increase the percentage of live cells post-injection into ischemic tissue for therapeutic repair.

Self-assembling oligopeptides are attractive candidates for biocompatible three-dimensional scaffolds that can support and regulate cell function as a substitute for their normal physiological microenvironment. In particular, peptide-based hydrogel materials are generally cytocompatible, and are inherently more physiologically relevant as they allow for cellular remodeling and can promote cell viability. However, there are relatively few examples of these oligopeptides that exist beyond the derivation or analogs of the well-established diphenylalanine peptide sequence, which are able to form robust hydrogel systems, but their inherent hydrophobicity of the sequence limits their solubility and thereby, extension of their application.

Also, the relatively few oligopeptides which have been studied are derived by sequence mapping onto relevant biological systems that have already been known to self-assemble into a variety of nanostructures. Thus, there exists an ongoing need for self-assembling oligopeptides that can form biocompatible three-dimensional scaffolds that address these problems.

### SUMMARY

In an aspect, the disclosure provides a peptide comprising or consisting of SEQ ID NO: 1 (KFFFL consisting of L amino acids) or 2 (KFFFL consisting of D amino acids), or comprising or consisting of a sequence having at least 80% identity to SEQ ID NO: 1 or 2. The peptide may comprise an amidated C-terminus.

In a second aspect, the disclosure comprises a kit comprising an L-peptide and a D-peptide, the L-peptide comprising or consisting of SEQ ID NO: 1, or a sequence having at least 80% identity thereto, and the D-peptide comprising or consisting of SEQ ID NO: 2, or a sequence having at least 80% identity thereto. The L-peptide and D-peptide may each comprise an amidated C-terminus.

In a third aspect, the disclosure provides a hydrogel composition comprising an aqueous dispersion medium and at least one peptide selected from an L-peptide and a D-peptide, the L-peptide comprising or consisting of SEQ ID NO: 1, or a sequence having at least 80% identity thereto, and the D-peptide comprising or consisting of SEQ ID NO: 2, or a sequence having at least 80% identity thereto. In the composition, the hydrogel is formed by self-assembly of the at least one peptide in the aqueous dispersion medium. The hydrogel may comprise only the L-peptide, only the D-peptide, or both the L-peptide and the D-peptide. The L-peptide and the D-peptide may be at an L:D ratio of about 1:3 to about 3:1.

The aqueous dispersion medium may comprise one or more salts. The hydrogel composition may have a pH of about 7 to about 11. The at least one peptide may be in the hydrogel at an amount of at least about 3% w/v.

In embodiments, the at least one peptide forms a nanospiral structure.

The at least one peptide may comprise an amidated C-terminus.

In a fourth aspect, the disclosure provides a method of preparing any of the disclosed hydrogel compositions, the method comprising preparing a peptide solution comprising the at least one peptide dissolved in water; adding a salt buffer to the peptide solution; and adding a salt to the peptide solution and mixing the solution for at least about 30 minutes and until the peptide solution is at least about pH 7.

In a fifth aspect, the disclosure provides a pharmaceutical composition comprising any of the disclosed hydrogel compositions, and an active agent.

In a sixth aspect, the disclosure provides a method of treating a subject with an active agent, the method comprising administering any of the disclosed pharmaceutical compositions to the subject.

In a seventh aspect, the disclosure provides a method of treating damaged tissue in a subject, the method comprising contacting the damaged tissue with any of the disclosed hydrogel compositions. The hydrogel may be formed on the damaged tissue, or may be formed in vitro and transferred to the damaged tissue.

In an eighth aspect, the disclosure provides a method of culturing a cell, the method comprising contacting the cell with any of the disclosed hydrogel composition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show representative transmission electron microscopy (TEM) images comparing nanoscale morphologies at high (FIG. 1A) and low (FIG. 1B) resolution across 1.5% (w/v) L-, 3:1 L:D-, 1:1 L:D-, 1:3 L:D-, and D-KFFFL hydrogels (**). Only fibers are present in pure L-(SEQ ID NO: 1) and D-KFFFL (SEQ ID NO: 2), whereas 3:1 L:D- and 1:3 L:D-KFFFL contain fibers, plates, and spirals, and 1:1 L:D-KFFFL contains plates and spirals. Fibers are a commonly reported morphology, and plates have been reported previously in KYFIL hydrogels, however spirals have yet to be reported in peptide-based hydrogels composed of natural amino acids without a directed self-assembly method.

FIGS. 2A-2C show representative transmission electron microscopy (TEM) images of spiral structures in 1.5% (w/v) 3:1 L:D-, 1:1 L:D-, 1:3 L:D-KFFFL hydrogels (**) at a high (FIG. 2A) middle (FIG. 2B), and low (FIG. 2C) magnification. Spirals have close fiber packing and circularity, distinguishing them from typical fibrillar morphologies.

FIGS. 3A-3F. (FIG. 3A) Example of major and minor axis nanospiral measurements for nanospiral diameter calculations in (FIG. 3D), where diameter is defined as the average of the major and minor axis for well-formed spirals. (FIG. 3B) Example of fiber diameter measurements for fibers within nanospirals in (FIG. 3E). (FIG. 3C) Example of fiber diameter measurements for non-nanospiral fibers in (FIG. 3F). (FIG. 3D-3F) Scatter plots combined with box and whisker plots. Scatter plots show individual data points, where duplicates at two significant figures are displaced to the right. Box and whisker plots show the distribution of data, with the median value represented at the midline, and the mean value represented as “x”. (FIG. 3D) Nanospiral diameter comparison across the three mixtures of 3:1 L:D- (N=20 spirals), 1:1 L:D- (N=68 spirals), and 1:3 L:D- (N=21 spirals) KFFFL. (FIG. 3E) Nanospiral layer thickness diameter comparison across the three mixtures of 3:1 L:D- (N=11 spirals), 1:1 L:D- (N=11 spirals), and 1:3 L:D- (N=17 spirals) KFFFL. (FIG. 3F) Nanofiber diameter measurements for L-, 3:1 L:D-, 1:3 L:D-, and D-KFFFL (N=150 fibers each). Spiral structures are ~100-500 nm in diameter with layers approximately 3-4 nm in diameter, measuring smaller than non-spiral fibers with diameters ranging from 7-15 nm.

FIGS. 4A-4E show simulations of KYFIL (SEQ ID NO: 3, L-KYFIL; SEQ ID NO: 4, D-KYFIL) and KFFFL conducted at various ratios of L- and D-peptide. (FIG. 4A) Clustering of KYFIL and KFFFL for 1:1 L:D-peptide at 0, 100, and 200 ns of L-peptide (blue) and D-peptide (red). Cluster formation occurs between L- and D-peptide, indicating interactions between enantiomers. (FIG. 4B) Total number of hydrogen bonds over 200 ns. (FIG. 4C) Total number of II-II stacking interactions over 200 ns. (FIG. 4D) Average total number of hydrogen bonds over the last 20 ns of (FIG. 4B). (FIG. 4E) Average total number of II-II stacking interactions over the last 20 ns of (FIG. 4C). Error bars in (FIG. 4D) and (FIG. 4E) represent standard deviation. Across different ratios of L- and D-peptide, KYFIL and KFFFL have similar amounts of hydrogen bonding, yet II-II stacking interactions in KFFFL are approximately 2 to 4 times greater than KYFIL.

FIGS. 5A-5E show transmission electron microscopy (TEM) images for 1.5% (w/v) KFFFL hydrogels (**) displaying atypical morphologies including nanospirals without well-defined edges, fiber packing, or diameters: (FIG. 5A) Rosettes, (FIG. 5B) incomplete nanospirals, (FIG. 5C) broken spirals, (FIG. 5D) mismatched, and (FIG. 5E) magnification of mismatched image section in (FIG. 5D) with groups of spiral nanofibers outlined in gray. These images highlight the heterogeneity in nanospiral appearance.

FIGS. 6A-6C show KFFFL hydrogel formulation and rheology. (FIG. 6A) Formulation process for peptide in ultrapure water (*) and with salt addition and pH correction (**). PBS=Phosphate Buffered Saline. (FIG. 6B) Images of 1.5% (w/v) L-, 3:1 L:D-, 1:1 L:D-, 1:3 L:D-, and D-KFFFL hydrogel formation (**). Most groups form hydrogels that pass the inversion test as early as 30 minutes after pH correction. 1:1 L:D-KFFFL is the exception, taking ~1 hour for partial gelation to occur. (FIG. 6C) Storage modulus (G′) and loss modulus (G″) measurements for 1.5% (w/v) of each L:D ratio forming KFFFL hydrogels (**). As stereocomplexation increases, G′ and G″ decrease, indicating potential differences in underlying morphologies affecting hydrogel mechanical properties. N=3 independently prepared samples, where error bars represent the standard deviation.

FIGS. 7A-7B show 1.5% (w/v) KFFFL in ultrapure water. (FIG. 7A) Pure L- and D-KFFFL are soluble in solution, while mixtures of L- and D-KFFFL form opaque solutions. (FIG. 7B) Turbidity of each experimental group in (FIG. 7A) Turbid solutions suggest stereocomplexation occurs in L- and D-KFFFL mixtures prior to salt addition and pH correction.). N=2 independently prepared samples, 2 measurements per sample, where error bars represent standard deviation across 4 measurements.

FIGS. 8A-8B show FTIR spectra of KFFFL, where shaded region is from 1620-1640 cm⁻¹. (FIG. 8A) In ultrapure water (*) all L:D-KFFFL mixtures contain an amide I absorbance peak at ~1630 cm⁻¹, indicating the presence of β-sheets. (FIG. 8B) After salt addition and pH correction (**), all formulations, including mixtures and L- and D-KFFFL independently, contain an amide I absorbance peak at ~1630 cm⁻¹. Together, this demonstrates that although β sheet content is present in formulations that form nanospirals, β sheet presence alone is not sufficient for nanospiral formation.

FIGS. 9A-9F show time dependence in nanospiral formation (**). Transmission electron microscopy (TEM) images at (FIG. 9A) ~30 minutes and (FIG. 9B) ~24 hours post-formulation in KFFFL hydrogels. Quantification of changes in (FIG. 9C) fiber diameter in L-KFFFL hydrogels, (FIG. 9D) nanospiral layer thickness in 1:1 L:D-KFFFL hydrogels, (FIG. 9E) nanospiral layer thickness in 3:1 L:D-KFFFL hydrogels, and (FIG. 9F) nanospiral diameter in 3:1 L:D-KFFFL hydrogels at ~24 hours and ~30 minutes post-formulation. Although the data means from 30 minutes to 24 hours appear similar, at ~30 minutes post-formulation (**) there is greater variance compared to 24 hours, suggesting morphology formation may take longer than 30 minutes to reach thermodynamically stable structures.

FIG. 10 shows TEM images of 1.5% (w/v) L-KFFFL in 1×PBS at pH=7.4±0.2, showing fiber morphologies.

FIG. 11 shows TEM images of 1.5% (w/v) 3:1 _{L:D}-KFFFL in 1×PBS at pH=7.4±0.2, showing spiral, plates, and fiber morphologies. The top right image illustrates spiral structures stacked on top of fibers.

FIG. 12 shows TEM images of 1.5% (w/v) 1:1 _{L:D}-KFFFL in 1×PBS at pH=7.4±0.2, showing spiral, and plate morphologies.

FIG. 13 shows TEM images of 1.5% (w/v) 1:3 _{L:D}-KFFFL in 1×PBS at pH=7.4±0.2, showing spiral, plates, and fiber morphologies.

FIG. 14 shows TEM images of 1.5% (w/v) D-KFFFL in 1×PBS at pH=7.4±0.2, showing fiber morphologies.

FIG. 15 shows TEM images of 1.5% (w/v) 3:1 _{L:D}-KFFFL in 1×PBS at pH=7.4±0.2, showing spiral morphologies. Red letters denote spirals for which the major and minor axis were quantified, while blue lines indicate the regions where layer thickness measurements were performed.

FIG. 16 shows TEM images of 1.5% (w/v) 1:1 _{L:D}-KFFFL in 1×PBS at pH=7.4±0.2, showing spiral morphologies. Red letters denote spirals for which the major and minor axis were quantified, while blue lines indicate the regions where layer thickness measurements were performed.

FIG. 17 TEM images of 1.5% (w/v) 1:3 _{L:D}-KFFFL in 1×PBS at pH=7.4±0.2, showing spiral morphologies. Red letters denote spirals for which the major and minor axis were quantified, while blue lines indicate the regions where layer thickness measurements were performed.

FIGS. 18A-18D illustrate the comparison of major and minor axis (FIG. 3A) nanospiral diameter measurements for (FIG. 18A) 3:1 _{L:D}-KFFFL 30 minutes post-formulation, (FIG. 18B) 3:1 _{L:D}-KFFFL 24 hours post-formulation, (FIG. 18C) 1:1 _{L:D}-KFFFL 24 hours post-formulation, and (FIG. 18D) 1:3 _{L:D}-KFFFL 24 hours post-formulation. Similar major and minor axis measurements indicate nanospirals are circular, and therefore major and minor axes are averaged together for represented data in FIG. 3D.

FIGS. 19A-19C illustrate the comparison of spiral layer thickness measurement techniques for incomplete and complete nanospirals in 1:1 _{L:D}-KFFFL. (FIG. 19A) Measurements of individual spiral layers. (FIG. 19B) Measurements of average layer thicknesses, described in FIG. 3B. In both (FIG. 19A) and (FIG. 19B), thickness is approximately the same for complete and incomplete nanospirals. (FIG. 19C) Comparison of methods in (FIG. 19A) and (FIG. 19B) shows that average thickness measurements are slightly higher than individual measurements. Due to the need for high magnification images to reliably measure individual spiral layer thickness, the average method was used (FIGS. 3B and 3E).

FIG. 20 shows TEM images of rosettes. This abnormality was only found in 1.5% (w/v) 1:1 _{L:D}-KFFFL in 1×PBS at pH=7.4±0.2. No measurements were taken on spirals of this type.

FIG. 21 shows TEM images of conjoined spirals that take on the appearance of a palmier due to two nanospirals joined together. All images from 1.5% (w/v) peptide in 1×PBS mixtures at pH=7.4±0.2. Top left image of 3:1 _{L:D}-KFFFL at 30 minutes is a lower magnification of FIG. 4B. Top right image of 1:1 _{L:D}-KFFFL at 24 hours post-formulation. Bottom images are 1:1 _{L:D}-KFFFL at 24 hours post-formulation lower magnification (left) and higher magnification (right).

FIG. 22 shows TEM images of incomplete spirals. This abnormality was found in all 1.5% (w/v) _{L:D}-KFFFL in 1×PBS mixtures at pH=7.4±0.2. Red letters denote spirals for which the major and minor axis were quantified, while blue lines indicate the regions where layer thickness measurements were performed. No representative images of this abnormality are shown in 1:1 _{L:D}-KFFFL due to low resolution.

FIG. 23 shows TEM images of broken spirals. This abnormality was found in all 1.5% (w/v) _{L:D}-KFFFL in 1×PBS mixtures at pH=7.4±0.2. Blue lines indicate the regions where layer thickness measurements were performed. The major and minor axes of these spirals were not quantified. These nanospirals are likely present due processing, where TEM grid staining involves the use of a razor blade.

FIG. 24 shows TEM images of mismatched spirals. This abnormality was primarily found in 1.5% (w/v) 1:1 _{L:D}-KFFFL in 1×PBS mixtures at pH=7.4±0.2. Blue lines indicate the regions where layer thickness measurements were performed. The major and minor axes of these spirals were not quantified. No representative images of this abnormality are shown in 1:3 or 3:1 _{L:D}-KFFFL due to low resolution.

FIGS. 25A-25F illustrates time in nanospiral formation. Transmission electron microscopy (TEM) images at (FIG. 25A) ~30 minutes and (FIG. 25B) ~24 hours post-formulation in KFFFL hydrogels. Quantification of changes in (FIG. 25C) fiber diameter in _{L}-KFFFL hydrogels, (FIG. 25D) nanospiral layer thickness in 1:1 _{L:D}-KFFFL hydrogels, (FIG. 25E) nanospiral layer thickness in 3:1 _{L:D}-KFFFL hydrogels, and (FIG. 25F) nanospiral diameter in 3:1 _{L:D}-KFFFL hydrogels at ~24 hours and ~30 minutes post-formulation. Although the data means from 30 minutes to 24 hours appear similar, at ~30 minutes post-formulation there is greater variance compared to 24 hours, suggesting morphology formation may take longer than 30 minutes to reach thermodynamically stable structures.

FIG. 26 shows TEM images of nanospirals in 1.5% (w/v) 3:1 _{L:D}-KFFFL in 1×PBS mixtures at pH=7.4±0.2. Images taken 30 minutes after formulation.

FIGS. 27A-27E show the remaining results from simulations of KYFIL and KFFFL conducted at various ratios of _{L}- and _{D}-peptide. (FIG. 27A) Clustering snapshots after 200 ns simulation of KYFIL and KFFFL for _{L}-, 3:1 _{L:D}-, 1:1 _{L:D}-, 1:3 _{L:D}-, and _{D}-peptides. (FIG. 27B) Total number of clusters over 200 ns. (FIG. 27C) Total number of β-strands over 200 ns. (FIG. 27D) Average total number of clusters over the last 20 ns of (FIG. 27B). (FIG. 27E) Average total number of β-strands over the last 20 ns of (FIG. 27C). Error bars in (FIG. 27D) and (FIG. 27E) represent standard deviation. Results may imply the total number of clusters increases and the total number of β-strands decreases as _{L}- and _{D}-peptide ratio gets closer to 1:1.

FIG. 28 TEM images showing twisted fibers in 1.5% (w/v) 1:3 _{L:D}-KFFFL in 1×PBS at pH=7.4±0.2. These images were from samples formed using an alternate formulation procedure (FIG. 29).

FIG. 29A-29F show triplicate frequency sweeps for three independently prepared samples of 1.5% (w/v) KFFFL peptide in 1×PBS at pH=7.4±0.2: (FIG. 29A) _{L}-KFFFL, (FIG. 29B) 3:1 _{L:D}-KFFFL, (FIG. 29C) 1:1 _{L:D}-KFFFL, (FIG. 29D) 1:3 _{L:D}-KFFFL, and (FIG. 29E) _{D}-KFFFL. These hydrogels were subjected to oscillatory shear using a 25 mm diameter parallel plate geometry with a gap height of 500 μm. At room temperature, angular frequency was ramped logarithmically from 0.01 to 100 rad/s, with a strain of 0.1%. Solid symbols represent storage moduli and open symbols represent loss moduli. (FIG. 29F) Average storage moduli of all groups in A-E, with error bars representing standard deviation.

FIGS. 30A-30F show triplicate amplitude sweeps for three independently prepared samples of 1.5% (w/v) KFFFL peptide in 1×PBS at pH=7.4±0.2: (FIG. 30A) _{L}-KFFFL, (FIG. 30B) 3:1 _{L:D}-KFFFL, (FIG. 30C) 1:1 _{L:D}-KFFFL, (FIG. 30D) 1:3 _{L:D}-KFFFL, and (FIG. 30E) _{D}-KFFFL. These hydrogels were subjected to oscillatory shear using a 25 mm diameter parallel plate geometry with a gap height of 500 μm. At room temperature, strain was ramped logarithmically from 0.01 to 101% strain, with an angular frequency of 10 rad/s. Solid symbols represent storage moduli and open symbols represent loss moduli. (FIG. 30F) Average storage moduli of all groups from A-E, with error bars representing standard deviation. The vertical black bar denotes the maximum value the strain range of 0.01-0.05% strain, averaged together for FIG. 2C.

FIG. 31 shows a formulation procedure for KFFFL in water used for TEM imaging.

FIG. 32 shows inversion test images of 3% (w/v) 3:1 _{L:D}-, 1:1 _{L:D}-, and 1:3 _{L:D}-KFFFL in water. All mixtures pass the inversion test 10 minutes after mixing _{L}- and _{D}-peptide together.

FIG. 33 shows TEM images of 1.5% (w/v) _{L}-KFFFL in ultrapure water, showing blocky fiber-like morphologies.

FIG. 34 shows TEM images of 1.5% (w/v) 3:1 _{L:D}-KFFFL in ultrapure water, showing morphologies which were difficult to discern. Therefore, a lower w/v (%) was needed to view these morphologies.

FIG. 35 shows TEM images of 1.5% (w/v) 1:1 _{L:D}-KFFFL in ultrapure water, showing morphologies which were difficult to discern. Therefore, a lower w/v (%) was needed to view these morphologies.

FIG. 36 shows TEM images of 1.5% (w/v) 1:3 _{L:D}-KFFFL in ultrapure water, showing morphologies which were difficult to discern. Therefore, a lower w/v (%) was needed to view these morphologies.

FIG. 37 shows TEM images of 1.5% (w/v) _{D}-KFFFL in ultrapure water, showing blocky fiber-like morphologies.

FIG. 38 shows TEM images of 0.75% (w/v) 3:1 _{L:D}-KFFFL in ultrapure water, showing ribbon-like morphologies.

FIG. 39 shows TEM images of 0.75% (w/v) 1:1 _{L:D}-KFFFL in ultrapure water, showing morphologies which appeared to likely be ribbon-like, but were difficult to discern. Therefore, a lower w/v (%) was needed to view these morphologies.

FIG. 40 shows TEM images of 0.75% (w/v) 1:3 _{L:D}-KFFFL in ultrapure water, showing ribbon-like morphologies.

FIG. 41 shows TEM images of 0.50% (w/v) 1:1 _{L:D}-KFFFL in ultrapure water, showing ribbon-like, and potentially some fiber-like morphologies which appeared to be smaller in length.

FIG. 42 shows TEM images of 1.5% (w/v) 1:3 _{L:D}-KFFFL in 0.5×PBS at pH=7.4±0.2, showing spiral morphologies.

FIG. 43 shows formulation procedure that produced nanospirals (FIG. 30). 1× phosphate buffered saline (PBS) is added to peptide powder instead of ultrapure water. Images associated with this formulation strategy include: FIG. 1B 1:3 _{L:D}-KFFFL, 2B 1:3 _{L:D}-KFFFL, FIGS. 4C, 28, 30, 31, and 32. The formulation procedure in FIG. 7A was used to promote greater interaction between _{L}-KFFFL and _{D}-KFFFL prior to salt addition and was primarily used throughout this work.

FIG. 44 shows TEM images of nanospirals in 1.5% (w/v) 3:1 _{L:D}-KFFFL in 1×PBS at pH=7.4±0.2. These images were from samples formed using an alternate formulation procedure (FIG. 29)

FIG. 45 shows TEM images of nanospirals in 1.5% (w/v) 1:1 _{L:D}-KFFFL in 1×PBS at pH=7.4±0.2. These images were from samples formed using an alternate formulation procedure (FIG. 29).

FIG. 46 shows TEM images of nanospirals in 1.5% (w/v) 1:3 _{L:D}-KFFFL in 1×PBS at pH=7.4±0.2. These images were from samples formed using an alternate formulation procedure (FIG. 29)

FIGS. 47A-47B show FTIR Spectra in FIG. 9 with identified peaks. Peaks near ~1630 cm⁻¹ appeared alongside peaks between 1650-1700 cm⁻¹, indicating an antiparallel β-sheet conformation for enantiomeric peptide mixtures in water (A) and peptide hydrogels (B) after salt addition and pH-correction.

FIG. 48 shows the assessment of hydrogel mechanical properties over time. In this study, the storage modulus (G′) and loss modulus (G″) of 1.5 wt % L-KFFFL hydrogels were assessed at 30 min, 12 h, 24 h post-formulation. At each time point, a strain sweep was performed to how the gel responds to increasing stress, and a frequency sweep was performed to see how the gel reacts to faster or slower deformations. Overall, neither the storage nor loss modulus showed a significant increase—suggesting that the hydrogel's mechanical properties remain consistent within the 24-hour window.

FIG. 49 shows the recovery of 1.5 wt % KFFFL gels having varying L:D peptide ratios. The 100% L sample had the highest initial storage modulus, followed by 75% L, 25% L., 100% D, and finally 50% L. 75% L and 25% L have similar initial G′ and G″. The results suggest the recovery of the gel increases with the proportion of D-peptides. Values above 100% indicate that the gel became even stiffer than before the high strain.

FIG. 50 shows the stress relaxation of 1.5 wt % KFFFL gels having varying L:D peptide ratios. As with the recovery results, stress relaxation also appears to increase with the proportion of D-peptides.

### DETAILED DESCRIPTION

The present disclosure relates to novel self-assembling peptides. As demonstrated herein, present inventors discovered that the pentapeptides L-KFFFL and D-KFFFL, under certain conditions, are capable of self-assembling to form nanospirals, a unique nanomorphology that has not been previously observed without the use of directed assembly methods and non-natural/modified amino acids. The self-assembling pentapeptides, and peptides containing the peptides, may be used to form robust hydrogels. These hydrogels, which may be referred to herein as rapidly assembling pentapeptides for injectable delivery (RAPID) hydrogels are syringe-injectable and support cytocompatible encapsulation of cells, including oligodendrocyte progenitor cells (OPCs), as well as their proliferation and three-dimensional process extension.

### Peptides, Nucleic Acids, and Kits

In an aspect, the disclosure provides a peptide comprising the amino acid sequence set forth in SEQ ID NO: 1 (KFFFL, wherein each amino acid is an L amino acid, an “L-peptide”) or 2 (KFFFL, wherein each amino acid is a D amino acid, a “D-peptide”), or an amino acid sequence having at least 60%, 70%, 80%, or 90% identity to SEQ ID NO: 1 or 2. As used herein, the term “peptide” refers to a polymer of amino acid residues. The term may apply to amino acid polymers in which one or more amino acid residues is an artificial chemical analogue of a corresponding naturally occurring amino acid, as well as to naturally occurring amino acid polymers. Suitably, the methods disclosed herein may also comprise or employ variants and fragments of the disclosed amino acids. In embodiments, the peptide is capable of self-assembling and forming robust hydrogels (e.g., in aspects, upon mixing under physiological conditions as described herein). In aspects, the present disclosure is directed to a nucleic acid sequence (e.g., DNA or RNA) encoding one or more peptides of the present disclosure. In some embodiments, the peptide is a 6-mer, 7-mer, 8-mer, 9-mer, or 10-mer peptide comprising multiple repeats of SEQ ID NO: 1 or 2, or an amino acid sequence having at least 60%, 70%, 80%, or 90% identity to SEQ ID NO: 1 or 2.

The terms “protein”, “polypeptide”, and “peptide” are used to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine). In exemplary embodiments, the C-terminus of any of the peptides disclosed herein are amidated.

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

As used herein, a “positively charged amino acid” includes lysine (K), arginine (R), and histidine (H). As used herein, an “aliphatic amino acid” includes alanine (A), isoleucine (I), proline (P), and valine (V). As used herein, an “aromatic amino acid” includes phenylalanine (F), tryptophan (W), tyrosine (Y), and histidine (H). As used herein, a “hydrophobic amino acid” includes glycine (G), alanine (A), valine (V), leucine (L), isoleucine (I), proline (P), phenylalanine (F), methionine (M), and tryptophan (W).

As used herein, a peptide or nucleic acid sequence is said to be “isolated” or “purified” when it is substantially free of cellular material when it is isolated from recombinant and non-recombinant cells, or free of chemical precursors or other chemicals when it is chemically synthesized. As such, an isolated or purified peptide or nucleic acid sequence is substantially free of other cellular material, or culture medium when produced by recombinant techniques, or substantially free of chemical precursors or other chemicals when chemically synthesized. A peptide or nucleic acid sequence that is substantially free of cellular material includes preparations of peptides having less than about 30%, 20%, 10%, 5%, 1%, or any value or range therebetween (by dry weight) of other proteins (e.g., contaminating proteins). When the peptides are recombinantly produced, optimally culture medium represents less than about 30%, 20%, 10%, 5%, 1%, or any value or range therebetween (by dry weight) of chemical precursors or non-protein-of-interest chemicals that are involved in the peptide or nucleic acid sequence synthesis.

“Percentage of sequence identity”, “percent similarity”, or “percent identity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. Embodiments described herein have at least: 75%, 80%, 85%, 86%, 87%, 88%, 89%, 90%, 91%, 92%, 93%, 94%, 95%, 96%, 97%, 98% or 99% identity compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

When homologous is used in reference to polypeptides, it is recognized that residue positions that are not identical can often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are known to those of skill in the art. The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

In embodiments, amino acid residues which are not believed to be essential for the functioning of the presently disclosed polypeptides may be substituted either conservatively or non-conservatively, and such amino acid substitutions would likely not significantly diminish the functional properties of the polypeptides.

A “variant” peptide is intended to mean a peptide derived from the presently disclosed peptides by deletion or addition of one or more amino acids at one or more internal sites in the native protein and/or substitution of one or more amino acids at one or more sites in the native protein. Variant peptide encompassed by the present disclosure continue to possess the desired properties of the present disclosure, that is, self-assembling pentapeptides capable of forming robust nanofiber hydrogels upon mixing under physiological conditions as described herein. Such variants may result from, for example, human manipulation. Variants will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the peptides of the present disclosure.

Peptides of the instant disclosure can be produced either from a nucleic acid sequence, or by the use of standard molecular biology techniques, such as recombinant techniques, mutagenesis, synthetic peptide production techniques, or other known means in the art. Accordingly, also disclosed herein are nucleic acids encoding the peptides of SEQ ID NO: 1 and SEQ ID NO: 2.

An isolated peptide can be purified from cells that naturally express it, purified from cells that have been altered to express it (recombinant), or synthesized using known protein synthesis techniques. In aspects, peptides of the instant disclosure are produced by recombinant DNA or RNA techniques. In aspects, a peptide can be produced by expression of a recombinant nucleic acid in an appropriate host cell. For example, a nucleic acid molecule encoding the peptide is cloned into an expression cassette or expression vector, the expression cassette or expression vector introduced into a host cell and the polypeptide expressed in the host cell. The peptide can then be isolated from the cells by an appropriate purification scheme using standard protein purification techniques. Alternatively, a peptide can be produced by a combination of ex vivo procedures, such as protease digestion and purification. Further, peptides can be produced using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

The terms “polynucleotide,” “polynucleotide sequence,” “nucleic acid,” and “nucleic acid sequence” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

In another aspect, provided herein is a kit comprising an L-peptide comprising or consisting of SEQ ID NO: 1 or a sequence having at least 80% identity thereto; and a D-peptide comprising or consisting of SEQ ID NO: 2 or a sequence having at least 80% identity thereto. The L-peptide and the D-peptide may be provided in separate containers.

### Hydrogel Compositions

In another aspect, the disclosure provides a hydrogel composition comprising at least one of the peptides disclosed herein, which peptides self-assemble to form the hydrogel. These hydrogels are useful in various applications, including but not limited to, scaffolds for tissue engineering, 2-dimensional (2-D) and 3-dimensional (3-D) cell cultures, drug delivery and encapsulation of therapeutic agents (cells, molecules, drugs, compounds), injectables (including those that gel in situ, such as hemostatic compositions), hemostatic agents, wound dressings, pharmaceutical carriers or vehicles, cell transplantation, cell storage, virus culture, and virus storage. It is to be understood that at least one peptide refers to at least one peptide having a distinct sequence. The at least one peptide comprises a plurality of peptides having that/those distinct sequence(s).

The hydrogel compositions may comprise (a) an L-peptide comprising or consisting of SEQ ID NO: 1, or a sequence having at least 60%, 70%, 80%, or 90% identity thereto, and/or (b) a D-peptide comprising or consisting of SEQ ID NO: 2, or a sequence having at least 60%, 70%, 80%, or 90% identity thereto.

In some embodiments, the hydrogel composition comprises only L-peptides. In other embodiments, the hydrogel composition comprises only D-peptides.

As demonstrated in the Examples, the inventors discovered that mixing L- and D-KFFFL together enabled the formation of robust hydrogels that exhibit a unique nanospiral morphology. Thus, in some embodiments, the hydrogel composition comprises L-peptides and the D-peptides. The L-peptides and D-peptides may be present in the composition and/or hydrogel in any suitable amount. The L-peptides and D-peptides may be present at an L-peptide: D-peptide (L:D) ratio of about 1:10, about 1:5, about 1:4, about 1:3, about 1:2, about 1:1, about 2:1, about 3:1, about 4:1, about 5:1, or about 10:1, or at an L:D ratio within a range bounded by any of the foregoing. In exemplary embodiments, the L-peptides and D-peptides are present at an L:D ratio of about 1:3 to about 3:1, or about 1:1.

The hydrogel composition may comprise an aqueous dispersion medium. Any suitable aqueous dispersion medium may be used. In some embodiments, the aqueous dispersion medium is physiologically acceptable. As used herein, “physiologically acceptable” in relation to the aqueous dispersion medium, refers to any suitable solution that is capable of conferring biologically acceptable conditions on the presently disclosed peptides such that are capable of self-assembly (i.e. with each other) resulting in gelation to form the hydrogel. Examples of suitable solutions will be known to the skilled technician, and may comprise a physiological buffer, such as saline or other buffered aqueous solutions.

In some embodiments, the aqueous dispersion medium comprises one or more salts. Any suitable salt may be used, including without limitation, Na₂PO₄, KH₂PO₄, NaCl, KCl, and any combination thereof. In some embodiments, the aqueous dispersion medium may comprise phosphate buffered saline, DMEM and other appropriate aqueous medium as known to one skilled in the art. For example, OPC media (e.g., DMEM with high glucose, 4 mM L-glutamine, 1 mM sodium pyruvate (Life Technologies) with N2 and B27 supplement (Life Technologies), C2C12 media (e.g., DMEM with 10% Fetal Bovine Serum, and 1% penicillin-streptomycin), hMSC media (e.g., DMEM with 20% Fetal Bovine Serum, and 1% penicillin-streptomycin), MLF media (e.g., DMEM with high glucose, 4 mM L-glutamine, 1 mM sodium pyruvate (Life Technologies) and supplemented with 1% penicillin-streptomycin (Life Technologies) and 20% Fetal Bovine Serum), and iPSC-derived NSC media (DMEM/F12+Glutamax with N2, B27 without vitamin A, 1 μg/mL), or the like are all appropriate.

As used herein, the term “biologically acceptable” means that the hydrogel of the instant disclosure is substantially stable under in vivo conditions, e.g., pH, temperature, and/or ionic strength as found in vivo. In aspects, the aqueous dispersion medium has a pH from about 6 to about 11, from about 7 to about 11, from about 7 to about 10, from about 9 to about 11, from about 10 to about 11, from about 6 to about 8, from about 7 to about 8, from about 7.3 to about 7.5, about 7.4, or about 10.6, or a pH within a range bounded by any of the foregoing. In aspects, the hydrogel itself has a pH from about 6 to about 11, from about 7 to about 11, from about 7 to about 10, from about 9 to about 11, from about 10 to about 11, from about 10.4 to about 10.7, from about 6 to about 8, from about 7 to about 8, from about 7.3 to about 7.5, about 7.4, or about 10.6, including any values or ranges therebetween.

As used herein, a “hydrogel” refers to a gel in which water is the major dispersion medium. Preferably, the water disperses the components of the hydrogel, e.g., the peptides as disclosed herein. Preferably, the hydrogel comprises at least 80% (w/w) water, more preferably, at least 85% (w/w) water, and more preferably, at least 90% (w/w), even more preferably, at least 95% (w/w) water.

The disclosed hydrogels may comprise the at least one peptide in any suitable amount. In some embodiments, the at least one peptide is present in the hydrogel at a level of from about 0.1% w/v to about 5% w/v, including every value and range therebetween based upon the total weight of the hydrogel. In some embodiments the at least one peptide is present in said hydrogel at a level of from about 1.0% w/v to about 3.5% w/v, including every value and range therebetween based upon the total weight of the hydrogel. In some embodiments, the at least one peptide is present in said hydrogel at a level of from about 1.5% w/v to about 3.0% w/v, including every value and range therebetween based upon the total weight of the hydrogel. In embodiments, the peptide is in the hydrogel at an amount of at least about 3% w/v.

The presently disclosed pentapeptides make up the 3-dimensional nanofibrous network of the hydrogel structure. Thus, in some embodiments, the hydrogels comprise nanospirals formed by the peptides. The nanospirals may have a diameter of about 10 nm, 50 nm, 100 nm, 150 nm, 200 nm, 250 nm, 300 nm, 350 nm, 400 nm, 450 nm, 500 nm, or 75 nm, or a diameter within a range bounded by any of the foregoing. In some embodiments, the nanospirals have a diameter of about 100 nm to about 500 nm. In some embodiments, the nanospirals have an average diameter of about 230 nm. The nanospirals may comprise layers spaced at a regular interval. In some embodiments, the layers have a thickness of about 3-4 nm. The hydrogel may also exhibit additional morphologies. In some embodiments, the hydrogel further comprises nanofibers and/or nanoplates.

As demonstrated in the Examples, the disclosed peptides can be used to form robust hydrogels. In some embodiments, the hydrogel has a shear storage modulus (G′) of at least about 50 Pa. The hydrogel may have a shear storage modulus of about 50 Pa, about 100 Pa, about 500 Pa, about 1,000 Pa, about 5,000 Pa, about 10,000 Pa, about 15,000 Pa, about 20,000 Pa, about 25,000, about 30,000 Pa, about 35,000 Pa, about 40,000 Pa, about 45,000 Pa, or about 50,000 Pa, or a shear storage modulus within a range bounded by any of the foregoing. In some embodiments, the hydrogel has a shear storage modulus 50 Pa to about 30,000 Pa, including every value and range therebetween. The shear storage moduli of the disclosed hydrogels may be tuned to a desired application of the hydrogel by adjusting the peptide concentrations. By way of example, for an injectable hydrogel, the hydrogel matrix may have a storage modulus of from about 50 Pa to about 3,000 Pa and preferably from about 70 Pa to about 1,000 Pa. In embodiments in which a very strong hydrogel is desired, such as for scaffolding, a hydrogel of the instant disclosure may have a storage modulus of at least about 500 Pa, or from about 100 Pa to about 10,000 Pa, or from about 1,000 Pa to about 30,000 Pa. These gel strengths may be based upon a neutral pH (about 7-about 7.4) and a temperature of about room temperature (aka “ambient temperature” or about 20-25° C.). In embodiments, the hydrogels of the instant disclosure are water-soluble. As used herein, “water-soluble”means the gels can be diluted with water or an aqueous polar solution (e.g., PBS, DMEM media, or the like) after formation.

The hydrogels may be characterized by having a “reversible” hydrogel matrix, which means that the 3-dimensional nanofibrous matrix is shear thinning (i.e., the viscosity decreases with an increase in the rate of shear stress applied to the gel), but recovers quickly after gel destruction. As such, in some embodiments, the hydrogel is a shear-thinning hydrogel. After gel destruction by subjecting the gel to a sufficient mechanical force (e.g., shear thinning, by, e.g., injection through a needle), the hydrogels max have a % recovery of at least about 60%, preferably at least about 80%, more preferably at least about 90%, and even more preferably about 100% in less than about 10, 9, 8, 7, 6, 5, 4, 3, 2, or 1 minutes (after removing the shear stress from the destroyed gel). In aspects, after 100% strain, the hydrogels recover gel behavior (G′>G″) in less than about 1 minute, 30 seconds, or 15 seconds. As used herein, the “% recovery” of the hydrogel is the percentage of the original storage modulus (i.e., before gel destruction) achieved by the gel after destruction and re-hydrogelation. Thus, shear-thinning only temporarily destroys the gel structure or architecture. As is understood by a person of skill in the art, shear-thinning may be carried out using various mechanical forces that impose a shear strain or shear stress on the hydrogel, such as pipetting, centrifugation, vibration, injection, spraying, filtration, and the like. As noted above, reassembly of the gel or hydrogelation reoccurs quickly after shear thinning and destruction of the gel structure (i.e., after removal of or stopping the application of mechanical force to the destroyed gel). This recovery property may also persist even after destroying the gel structure multiple times. The destroyed matrix after shear thinning can also be diluted with solvent to a substantially liquid solution (i.e., G′<0.2 Pa) to stop the recovery process.

In another aspect, provided herein is a pharmaceutical composition comprising the hydrogel composition disclosed herein, and an active agent. Active agents include, without limitation, small molecule drugs, biologics (e.g., enzymes, antibodies, other proteins and peptides, polynucleotides), and cells. The hydrogel may serve as a vehicle for delivering the active agent. The pharmaceutical composition may comprise a further pharmaceutically acceptable carrier. The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants.

As used herein, the terms “small molecular therapeutic agent”, “small molecule compound”, and “small molecule drug” refer to a chemical compound or pharmaceutically acceptable salt thereof having a therapeutic effect and/or enhancing the therapeutic effect of an immunotherapy. Small-molecule drugs are typically comprised of 20 to 100 atoms and have a molecular mass of less than 1000 g/mol or 1 kilodalton [kDa]. Small-molecules drugs can typically be administered by a variety of routes (including orally) and can pass through cell membranes to reach intercellular targets.

In another aspect, provided herein is a cell-supporting medium comprising a hydrogel composition as disclosed herein.

The disclosure also provides a liquid hydrogel precursor composition comprising: an aqueous dispersion phase comprising an aqueous dispersion medium; and at least one peptide as disclosed herein, wherein said composition is capable of being induced to form a hydrogel by self-assembly of said at least one peptide in said aqueous dispersion phase.

The disclosed hydrogel compositions, liquid hydrogel precursor compositions, pharmaceutical compositions, and cell-supporting mediums may comprise one or more substances, which may also act as lubricants, solubilizers, suspending agents, fillers, glidants, compression aids, or binders. The hydrogel compositions can be used as encapsulating materials. Liquid vehicles are used in preparing solutions, suspensions, emulsions, syrups, elixirs and pressurized compositions. The hydrogel compositions, liquid hydrogel precursor compositions, or pharmaceutical compositions may be dissolved or suspended in a pharmaceutically acceptable liquid vehicle such as water, an organic solvent, a mixture of both or pharmaceutically acceptable oils or fats. The liquid vehicle may contain other suitable pharmaceutical additives such as solubilizers, emulsifiers, buffers, preservatives, sweeteners, flavoring agents, suspending agents, thickening agents, colours, viscosity regulators, stabilizers or osmo-regulators. Suitable examples of liquid vehicles for oral and parenteral administration and implants include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration.

The liquid vehicle for pressurized compositions can be halogenated hydrocarbon or other pharmaceutically acceptable propellent.

In another aspect, the disclosure provides a nanospiral comprising at least one of (a) L-peptides comprising or consisting of the amino acid sequence set forth in SEQ ID NO.: 1, or an amino acid sequence having at least 60%, 70%, 80%, or 90% identity to SEQ ID NO: 1, and (b) D-peptides comprising or consisting of the amino acid sequence set forth in SEQ ID NO.: 2, or an amino acid sequence having at least 60%, 70%, 80%, or 90% identity to SEQ ID NO: 2. The L-peptides and D-peptides may be present in any suitable amount, and the nanospiral may have any suitable diameter and layer thickness, described herein. In some embodiments, the L-peptides and D-peptides are present at an L:D ratio of about 1:3 to about 3:1, or about 1:1. In some embodiments, the nanospiral has a diameter of about 100 nm to about 500 nm, and/or an average diameter of about 230 nm.

### Methods of Forming Hydrogels

In another aspect, the disclosure provides methods of forming the disclosed hydrogel compositions. The methods comprise (a) preparing a peptide solution comprising the at least one peptide dissolved in water; (b) adding a salt buffer to the peptide solution; and (c) adding at least one of a salt and an acid to the peptide solution for at least about 30 minutes and until the peptide solution is at least about pH 6. In embodiments, the pH is adjusted to at least about 7. The pH may be suitably adjusted to a pH from about 6 to about 11, from about 7 to about 11, from about 7 to about 10, from about 9 to about 11, from about 10 to about 11, from about 6 to about 8, from about 7 to about 8, from about 7.3 to about 7.5, about 7.4, or about 10.6, including any values or ranges therebetween. In embodiments, the salt is sodium hydroxide (NaOH). In exemplary embodiments the salt buffer is phosphate buffered saline. Acids suitable for pH adjustment include, without limitation, HCl, formic acid (HCOOH), acetic acid (CH3COOH), HBr, and nitric acid (HNO3) until such pH is achieved. Further, as the hydrogels of the instant disclosure are substantially stable under in vivo conditions, e.g., pH, temperature, and/or ionic strength as found in vivo, the hydrogel may be formed at a treatment site of an individual (e.g., a mammal, such as, but not limited to a human). For example, a liquid hydrogel precursor composition may be delivered to a treatment site of an individual, where due to the pH of the treatment site (e.g., pH≈7.4), the liquid hydrogel precursor composition will form a hydrogel. In some cases, the hydrogel forms when G′ (storage modulus) is greater than G″ (storage loss).

### Methods of Use

As previously described, the disclosed hydrogels and related compositions as described herein are useful in various applications, including but not limited to, scaffolds for tissue engineering, 2-dimensional (2-D) and 3-dimensional (3-D) cell cultures, drug delivery and encapsulation of therapeutic agents (cells, molecules, drugs, compounds), injectables (including those that gel in situ, such as hemostatic compositions), hemostatic agents, wound dressings, pharmaceutical carriers or vehicles, cell transplantation, cell storage, in vitro toxicity testing, virus culture, and virus storage.

In an aspect, the disclosure provides a method of treating a medical condition characterized by tissue damage and/or tissue loss in a subject in need thereof. The methods comprise forming a hydrogel composition as disclosed herein, wherein said forming is conducted (a) at a treatment site of the subject, or (b) in vitro followed by transferring said hydrogel to said treatment site. The hydrogel composition may be formed using any suitable method disclosed herein. Medical conditions characterized by tissue loss/damage include, but are not limited to, the treatment of wounds and tissue degenerative disorders (including neurodegenerative disorders, intervertebral disorders, muscle atrophy, kidney disorders, or cartilage or bone disorders.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process. As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a cell or subject by any suitable route for delivery of the therapeutic agent to the desired location.

As used herein, “preventative,” “preventing,” “prevent” and the like refer to partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

The terms “subject” and “patient” are used herein interchangeably to refer to a mammal to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. In embodiments, the subject is a human.

In the methods for treating a condition characterized by tissue loss/damage, the hydrogel or related composition as disclosed herein may comprise a cell. The cell may be of any cell type and is not particularly limited, and may include, e.g., epithelial cells, neurons, endothelial cells, osteoblasts, chondrocytes, fibroblasts, smooth muscle cells, osteoclasts, keratinocytes, nerve progenitor cells, stem cells, macrophages and other immune cells, and/or islet cells. As is understood by a person of skill in the art, the type of cell(s) included will depend on the type of tissue being repaired/regenerated.

In another aspect, the disclosure provides a method of delivering the hydrogel composition disclosed herein comprising an active agent to a subject in need thereof. As previously described, any suitable active agent may be delivered, including, without limitation, therapeutics such as small molecule drugs and/or biologics (e.g., enzymes, proteins, polynucleotides, and cells).

The hydrogels and/or hydrogel compositions may administered by any suitable means, including, without limitation, oral, rectal, nasal, systemic, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural) administration. In embodiments, the hydrogel compositions are administered topically. In embodiments, the hydrogel compositions are administered by injection, e.g. intravenous (bolus or infusion) or subcutaneous (bolus or infusion) injection. Further, the disclosed hydrogels and/or hydrogel compositions may also be incorporated within a slow or delayed release device. Such devices may, for example, be positioned on or adjacent the area to be treated, for example by implantation, and the hydrogel and related compositions of the present disclosure (and/or active agent associated therewith) may be released over weeks or even months. As is understood by a person of skill in the art, such devices may be particularly advantageous when long-term treatment with the medicament is required and which would normally require frequent administration (e.g. at least daily injection or implant).

The hydrogel compositions may be administered in a therapeutically effective amount. A “therapeutically effective amount” or “effective amount” is any amount which, when administered to a subject provides prevention and/or treatment of a specific medical condition.

The amount of the hydrogels and/or hydrogel compositions (and/or active agent associated therewith) required will be determined by its biological activity and bioavailability, which in turn depends on the mode of administration, the physicochemical properties of the medicament employed, and whether the hydrogel, compositions, cell-supporting medium, or medicament is being used as a monotherapy or in a combined therapy. The frequency of administration will also be influenced by the above-mentioned factors and particularly the half-life of the medicament within the subject being treated. Optimal dosages of the hydrogels and/or hydrogel compositions (and/or active agent associated therewith) to be administered may be determined by those skilled in the art, and will vary with the particular hydrogel, composition, and/or active agent in use, the strength of the preparation, the mode of administration, and the advancement of the disease condition. As is understood in the art, additional factors depending on the particular subject being treated will result in a need to adjust dosages, including subject age, weight, gender, diet, and time of administration.

The disclosed hydrogels and/or hydrogel compositions are capable of forming a hydrogel scaffold which is adapted to support cell growth. Thus, in another aspect, the disclosure provides a method of culturing a cell comprising contacting the cell with the hydrogel composition disclosed herein. Cell culture methodologies (culture time, temperatures, growth media etc.) are known in the art, and depend on the type of cell involved, and the final use of the cell-supporting medium (e.g., the hydrogel scaffold). In embodiments, cells are able to infiltrate the hydrogel, e.g., at a treatment site in an individual, and therefore form a 3D cell-supporting medium. Thus, the cell-supporting medium can serve to replace and/or repair damaged or lost tissue at the treatment site.

The cell may be of any cell type including, without limitation, epithelial cells, neurons, endothelial cells, osteoblasts, chondrocytes, fibroblasts, smooth muscle cells, osteoclasts, keratinocytes, nerve progenitor cells, stem cells, macrophages and other immune cells, and/or islet cells.

### Additional Terms

As used in this specification and the claims, the singular forms “a,” “an,” and “the” include plural forms unless the context clearly dictates otherwise. For example, the term “a protease” should be interpreted to mean “one or more proteases” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

The following examples are illustrative and are not intended to limit the scope of the claimed subject matter.

### EXAMPLES

### Example 1: Stereocomplexation of Pentapeptides Generates Supramolecular Nanospirals

Short self-assembling peptides are attractive biomaterials due to their low cost, and sequence tunability. Rationally determined residue selection enables noncovalent interactions between peptides that drive supramolecular assembly into fibers, ribbons, and rod-like morphologies, which in turn influence microscale and bulk material properties. Incorporating both L- and D-amino acids introduces stereocomplexation, offering a strategy to modulate nanostructure formation as well as biological and mechanical properties like enzyme-mediated degradation and hydrogel stiffness. A previously unknown nanospiral morphology emerges from mixing L- and D-KFFFL, a short peptide sequence comprising only three of the twenty naturally occurring amino acids. These nanospirals exhibit regularly spaced layers with diameters of ~3.4 nm, approximating peptide sequence length, and three times smaller than nanofibers formed from pure L- or D-KFFFL. Nanoplate structures also form in stereocomplexed samples, and salt plays a role in nanospiral, nanoplate, and nanofiber assembly. All hydrogels exhibit the presence of β sheets via Fourier transform infrared spectroscopy. Molecular dynamics simulations of KFFFL assembly reveal a 2- to 4-fold increase in pi-pi stacking compared to KYFIL, despite only a 1.5-fold increase in aromatic residues. These findings document a novel peptide nanospiral architecture and provide mechanistic insight into its formation. Supramolecular architectures such as the peptide nanospirals reported here open new avenues for biomolecular design, with broad implications for biomedical applications beyond the field of biomaterials.

### Introduction

The 1990 discovery of a short peptide sequence that gels on demand¹ initiated the development of peptide-based biomaterials, now widely applied in cell delivery,²⁻⁴ drug delivery,⁵⁻⁷ and regenerative medicine.⁸⁻¹⁰ Peptide self-assembly occurs via noncovalent interactions of hydrogen bonding, pi-pi stacking, hydrophobic interactions, electrostatic bonds, and van der Waals forces. 11,12 The relative presence of each of these interactions determines the thermodynamic stability of the system.^{11,13} Kinetic parameters of pH, temperature, concentration, and solvent choice modulate thermodynamic stability, driving the formation of secondary structures such as α-helices, β-sheets, etc.^{11,13} These secondary structures form larger, dynamic supramolecular structures of micelles, vesicles, and high aspect ratio fibrillar morphologies (fibers, ribbons, rods, tubes, etc.) ranging from the nanometer to micrometer scale.^{12,14-17}

Self-assembling peptides composed of naturally occurring amino acids are not recognized to form two-dimensional spiral morphologies.^{12,14-17} Spirals appear in many naturally occurring biological systems, from seashells to the cochlea of the human ear.¹⁸ Various synthetic chemical compounds with different assembly processes can create spiral structures on the nanometer scale. Nanospirals have been generated by spin coating diblock copolymer thin films on specified templates,^{19,20} self-assembling rosettes²¹ or rod-coil block molecules²²⁻²⁴, and using substrate-molecule interactions²⁵. Previous cases of spiral forming peptide sequences either require directed assembly methods or the use of chemical group(s) outside of the 20 naturally occurring amino acids. Compression driven interfacial assembly in Langmuir-Blodgett films produces spiral-like nanostructures in barbituric acid and anthracene derivatives²⁶⁻²⁸, and SARS-COV-2 derived peptides²⁹. Attaching an azobenzene group to the leading glycine of the silk-derived peptide GAGAGAGY (SEQ ID NO: 5) induces nanospiral formation.³⁰ High-temperature water vapor annealing drives ferrocene diphenylalanine to form Fibonacci spirals,³¹ which graphically represent the Fibonacci sequence, approximating golden (logarithmic) spirals³². In the presence of copper ions, L- and D-ferrocene diphenylalanine co-assemble into three-dimensional spheres of spiral architecture, which diminishes as the L:D-ratio diverges away from 1:1.33

Cases of nanospiral formation in chemical compounds involve aromatic groups.^{19-31,33} Notably, incorporating two or more phenylalanine residues in self-assembling peptide sequences is a well-established design strategy. Diphenylalanine is present in the core recognition motif of the β-amyloid (Aβ) peptide in Alzheimer's disease, wherein its self-assembly is driven by hydrogen bonding and pi-pi stacking between aromatic residues.^{34,35} The 42-residue Aβ peptide forms racemic fibrils³⁶ and the 7-residue Aβ fragment (16-22) yields rippled sheets³⁷ when L- and D-enantiomers are mixed. Triphenylalanine can form plate-like structures,³⁸ helical ribbons, toroids, and flower-shapes³⁹. Tetraphenylalanine with different N- and C-termini form a variety of structures, such as: nanoplates, fibrils, and star-like aggregates^{40,41} Alternating phenylalanine residues in two FKFE repeat motifs of opposite chirality assemble into helical tapes, whereas motifs with consistent chirality form fibers.⁴²

Combining L- and D-amino acids can alter nanostructures^{33,42-44} and modulate bulk properties in peptide-based biomaterials. The incorporation of D-amino acids have been employed to control enzymatic stability,⁴⁵ modulate degradation,⁴⁶ and induce both cytotoxic⁴⁶ or pro-regenerative⁴⁷ immune responses. Co-administration of Mito-FF with its D-form counterpart caused greater mitochondrial disruption and toxicity in cancer cells in vitro, and achieved higher tumor inhibition in vivo compared to either enantiomer alone.⁴⁴ Although this system did not gel, the racemic mixture formed 100 nm diameter fibers, whereas enantiomerically-pure formulations, produced 10 nm diameter fibers.⁴⁴ We recently reported a gel-forming peptide sequence, KYFIL, that forms fibers when L- and D-peptide are used independently, but assembles into nanoplate structures when mixed.⁴³ For racemic mixtures, shear moduli increased in water and decreased in phosphate-buffered saline compared to enantiomers, and D-peptide incorporation controlled proteolytic degradation.⁴³

In this work, we identify a self-assembling nanospiral morphology emerging from racemic mixtures of a pentapeptide composed of only three of the twenty canonical amino acids. By varying the L:D-peptide ratio and characterizing nanostructures with transmission electron microscopy (TEM), we quantify resulting morphology characteristics and assess formulation-dependent assembly behavior. We further compiled nanospiral formation mechanism evidence with molecular dynamics simulations, Fourier Transform Infrared Spectroscopy (FTIR), and comparison to another member of Rapidly Assembling Pentapeptides for Injectable Delivery (RAPID) hydrogel sequences, KYFIL.

### Results and Discussion

### Nanospiral Morphology Formation is a Stereochemistry-Driven Process

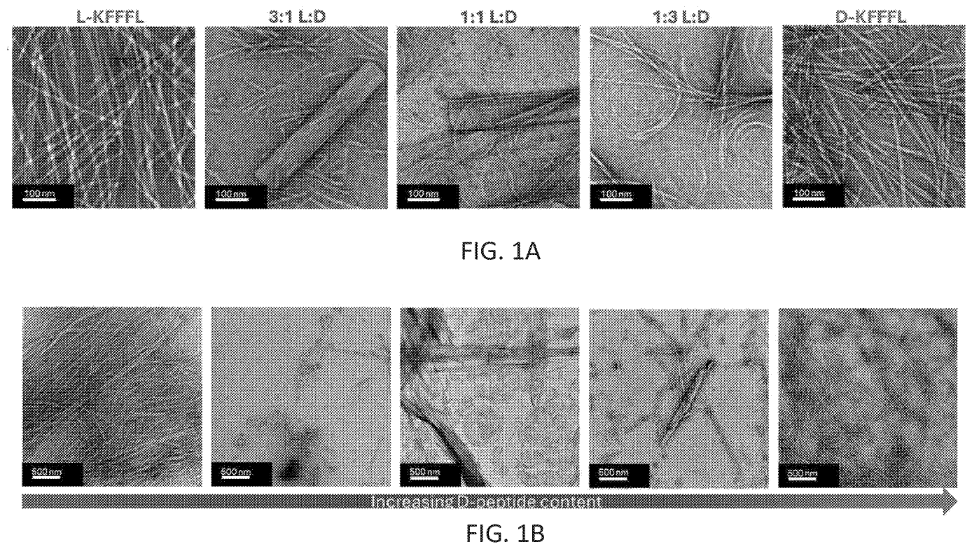

We hypothesized that mixing L- and D-KFFFL together would yield gels with nanomorphologies similar to KYFIL. At 1.5% (w/v), pure L- and D-KYFIL and their mixtures self-assemble into fibers and nanoplate structures.⁴³ Consistent with this hypothesis, L- and D-KFFFL mixtures at the same concentration induced self-assembly into fibers and nanoplates, and, unexpectedly, nanospirals (FIG. 1). Spiral morphologies are rarely documented in peptide-based material systems.^{12,14-17}

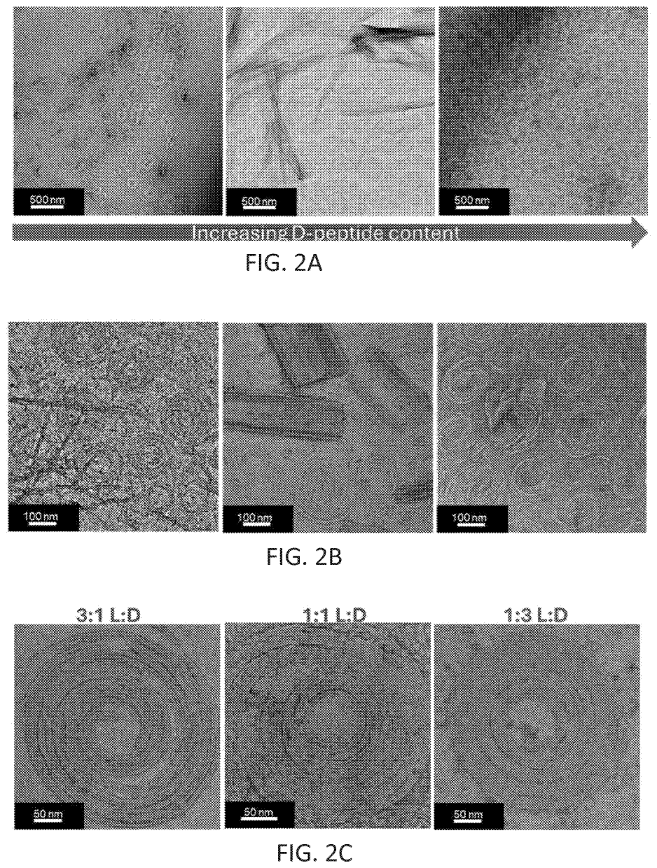

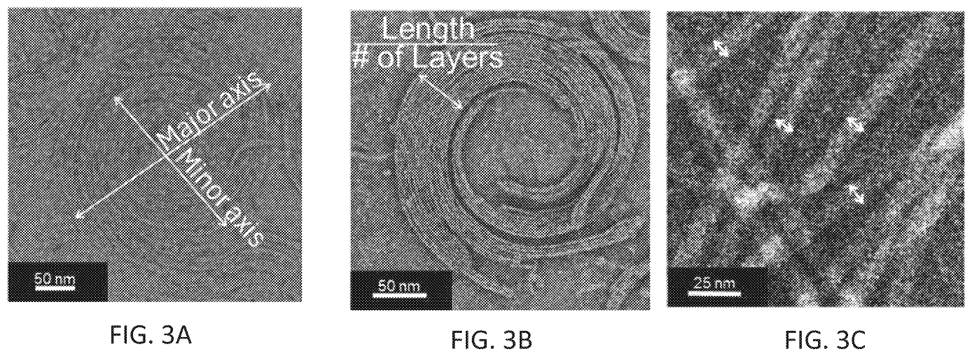

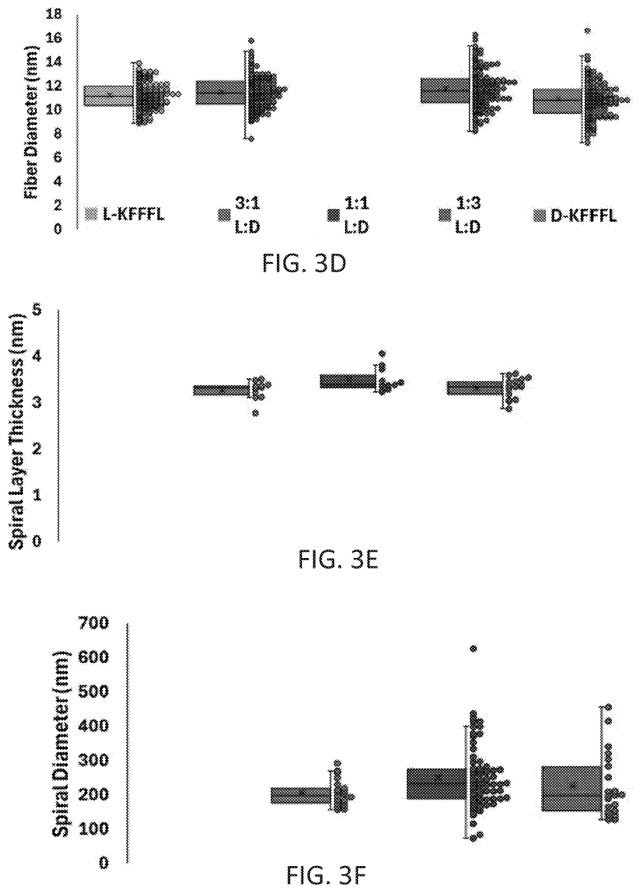

Nanospirals only appear in L- and D-KFFFL enantiomeric blends (FIG. 2). TEM imaging quantification revealed similar major and minor axis measurements (FIG. 3A), indicating a circular geometry with diameters ranging between ~100-500 nm, and an average diameter of 228+/−87 nm (FIG. 3D). Nanospirals contain regularly spaced layers (FIG. 3B) with thicknesses between ~3-4 nm, and an overall average diameter of 3.4 nm+/−0.2 nm (FIG. 3E). Fibers appeared in all groups, except 1:1 L:D-KFFFL (FIG. 2). These fibers were larger than nanospiral layers, with an average diameter of 11+/−1 nm and within the range of ~7-15 nm (FIGS. 3C and 3F). KYFIL fiber diameters also only appeared in L-, 3:1 L:D-, 1:3 L:D-, and D-formulations, but were larger, ranging from ~10-50 nm, with reported mean values of approximately 25 and 40 nm.^{43,48}

### KFFFL and KYFIL Molecular Dynamic Simulations

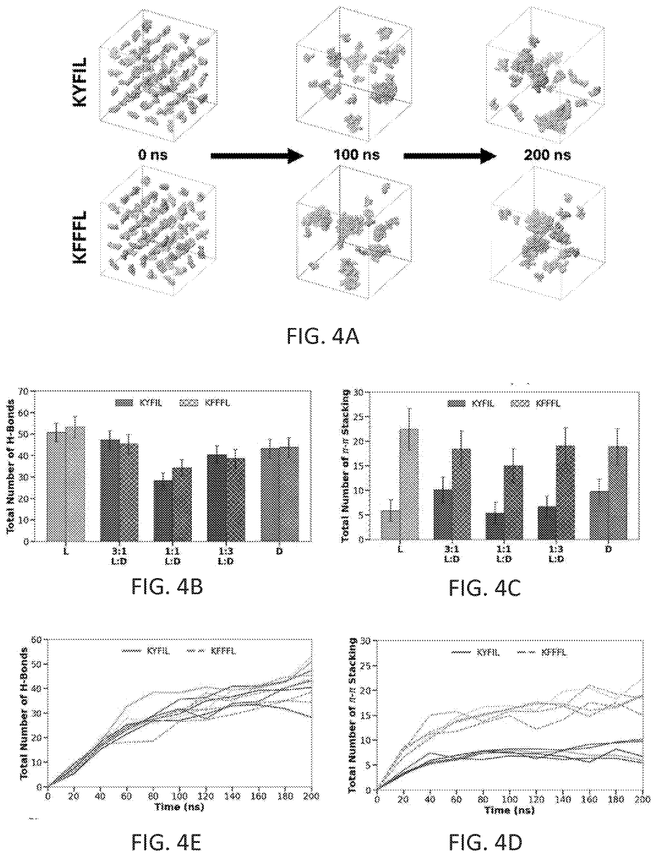

Morphology differences between L:D-peptide mixtures and pure L- and D-peptides suggests that peptides in mixtures form mixed clusters through mutual interactions rather than segregating and interacting exclusively with identical enantiomers. This behavior is supported by molecular dynamic simulations over 200 ns (FIG. 4A). Simulation results indicate insignificant differences in hydrogen bonding between KYFIL and KFFFL (FIGS. 4B and 4D). However, interestingly they reveal a 2 to 4-fold increase in pi-pi stacking across all groups despite only a 1.5-fold increase in aromatic residues (FIG. 4C, 4E, and Table 1). This information has been reported previously in molecular dynamics simulations comparing L-KYFIL to L-KFFFL, and supports the hypothesis that KFFFL fibers are smaller in diameter than KYFIL fibers due to increased order in the sequence imparted by changing out tyrosine (Y) and isoleucine (I) for two phenylalanine (F). Increased order could result in greater packing and alignment of peptides together to form fibers, and therefore greater amounts of pi-pi stacking. KFFFL and KYFIL sequences are approximately 3.4 nm in length, indicating that KFFFL fiber diameters are roughly three times their sequence length, whereas KYFIL fibers are at least six times larger.⁵³ Notably, nanospiral layer thickness is approximately equivalent to the sequence length of KFFFL and KYFIL. KYFIL fibers also exhibit noticeable twisting,^{43,48} compared to KFFFL fibers, which appear untwisted. We suspect that twisting occurs in KFFFL fibers, but is largely undetectable, as a few TEM images show twisted fibers. Although there are cases of overlapping standard deviations, other simulation results suggest no differences in the total number of clusters, and β-strands from KYFIL to KFFFL. They do however suggest that the total number of hydrogen bonds and β-strands may decrease as L:D-ratio converges towards 1:1, and conversely, that the total number of clusters increases (FIG. 4D). A higher number of clusters implies less interaction among peptides.

TABLE 1
Fold Increase in π-π Stacking from KYFIL to
KFFFL. Reported KYFIL and KFFFL
π-π Stacking

| L:D Ratio | KYFIL | KFFFL | Fold Increase |
|---|---|---|---|
| L-peptide | 5.89 ± 2.2 | 22.4 ± 4.2 | 3.7x |
| 3:1 L:D | 10.1 ± 2.5 | 18.4 ± 3.7 | 1.8x |
| 1:1 L:D | 5.40 ± 2.2 | 14.9 ± 3.5 | 2.8x |
| 1:3 L:D | 6.71 ± 2.2 | 19.0 ± 3.7 | 2.8x |
| D-peptide | 9.77 ± 2.5 | 18.9 ± 3.6 | 1.9x |

*values are the average ± standard deviation of the last 20 ns of simulation.

### Irregular Nanospiral Morphologies

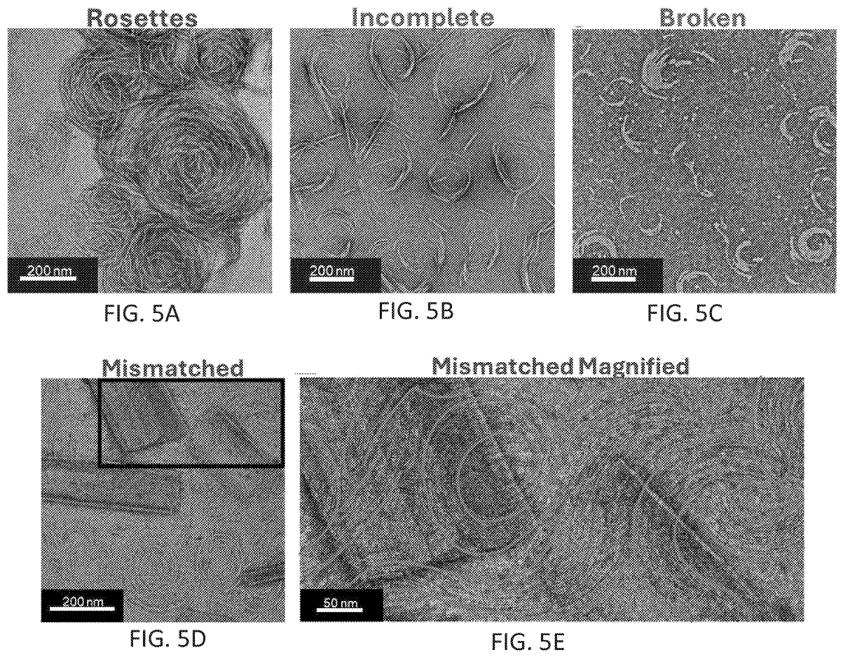

Imaging via transmission electron microscopy (TEM) revealed heterogeneity within spiral morphologies. In some cases, layer twisting within spirals was present, causing spirals to have a rosette appearance (FIG. 5A). These images appeared only in a select few 1:1 L:D-KFFFL images, however may imply that layer twisting occurs during spiral formation, but may still get trapped in a partially organized, layered structure. In other cases, only certain portions of spirals were visible, and they otherwise appeared incomplete (FIG. 5B). This is likely due to limitations of the TEM imaging technique and contrast differences. Fairly commonly, spirals appeared fragmented or broken (FIG. 5C), and thus only fiber spacing measurements within spirals were able to be collected from these images. This is expected from processing, where TEM grid staining involves the use of a razor blade. In many cases, there was mismatching of fibers resembling the fibers within spirals, often continuous and curved, but lacking the rolled-up appearance of a complete spiral (FIGS. 5D and 5E). Again, this could be contributed to processing, however, could also indicate a slightly different formation process.

### Nanospiral Morphology Mechanism

Interestingly, average spiral layer diameter is equivalent to peptide sequence length⁵³ and three orders of magnitude smaller than the average diameter of KFFFL fibers. In 3:1 and 1:3 L:D-KFFFL mixtures, these fibers have roughly the same diameter as fibers in pure L- and D-KFFFL (FIG. 3F). Spirals only appear in stereocomplexed gels, where nanospiral diameter is greater than fiber diameter, implying stereocomplexation enables packing of layers. These results suggest alternate mechanisms for spiral formation and fiber formation, as opposed to KFFFL fibers forming into spirals. In other words, nanospiral layers are not wound-up fibers. Furthermore, we can conclude spiral, plate, and fiber formation have independent assembly mechanisms, where plate and spiral formation are more thermodynamically favored, as evidenced by their appearance in 1:1 L:D-hydrogels without fibers.

Layer packing and curvature are two distinguishing features of spiral formation. For layer packing to occur, there first must be a step packing of peptides to form nanospiral layers, then a second step packing the layers together. Spirals exhibit packing of layers similar to grooved belts previously reported in bolaamphiphiles EFFFFE (SEQ ID NO: 6) and KFFFFK (SEQ ID NO: 7).⁵⁴ Attributed to their electrostatic interactions, these peptides form grooved belts when mixed in a 1:1 molar ratio in NH4OH.⁵⁴ In KYFIL hydrogels, when L- and D-hydrogels were formed separately, then mixed in a 1:1 ratio some packing and alignment was observed, although these structures were irregular and grooved belts were not present.⁴³ Based on this information, and without wishing to be bound by any particular theory the ordered layer packing and thereby peptide packing into layers may be driven by the three central phenylalanine in combination with stereocomplexation, where stereocomplexation may reduce the amount of electrostatic repulsion created by lysine between two different peptides.

Using Brownian dynamics simulations, curvature of spontaneous spiral formation in filaments was attributed to (1) flexibility in the filament, (2) the leading filament tip colliding with a different part on the same filament, and (3) a collision with enough force and volume exclusion for the tip to bend using enough force.^{30,55} These conditions were further expressed quantitatively, to show that spiral structures form with a high Péclet number (Pe) and low persistence length over length (ξ_{P}/L).^{30,55} Flexibility in nanospiral layers is likely achieved from flexibility between the three central phenylalanine, where tyrosine and isoleucine in KYFIL may prevent such flexibility due to increased steric hinderance. Brownian dynamics simulations were completed for a single filament only, but KFFFL spirals contain up to two sets of 9-10 leading layers (FIG. 3A, 1:1 L:D-KFFFL). It is possible spiral structures are the result of two sets of spiral layers colliding with subsequent parts of each other and wrapping around one another.

However, an alternative theory may suggest layers don't collide with one another but instead match side-by-side with one another first, then turn inwards, perhaps due to sequence amphiphilicity and/or offset phenylalanine pi-pi stacking in layers. Many spirals have a hollow center, which may arise from the first theory, where the colliding layers may be approaching with such force that they break, or maybe the second theory, where those layers are most difficult to turn inward, or perhaps this part of the spiral is prone to break during TEM processing methods. Mismatched irregular spiral structures (FIGS. 5D and 5E) also may occur from TEM processing methods, but alternatively may show that spiral layers prefer to pack together and display some curvature, regardless of spiral formation. This information could support either theory. In the first, perhaps the leading layers are colliding with the wrong part of other layers to promote spiral formation, or the lagging layers are blocked. In the second, blocked lagging layers could prevent spirals from curling together.

As with KYFIL, and other enantiomeric blends, the formation of different, larger morphologies has been reported.^{43,44} The mechanism to create such interesting nanospiral formations is unknown, but requires stereochemistry and sequence regularity to form characteristic features of packing and curvature.

### Rheological Properties of Stereocomplexed KFFFL Hydrogels

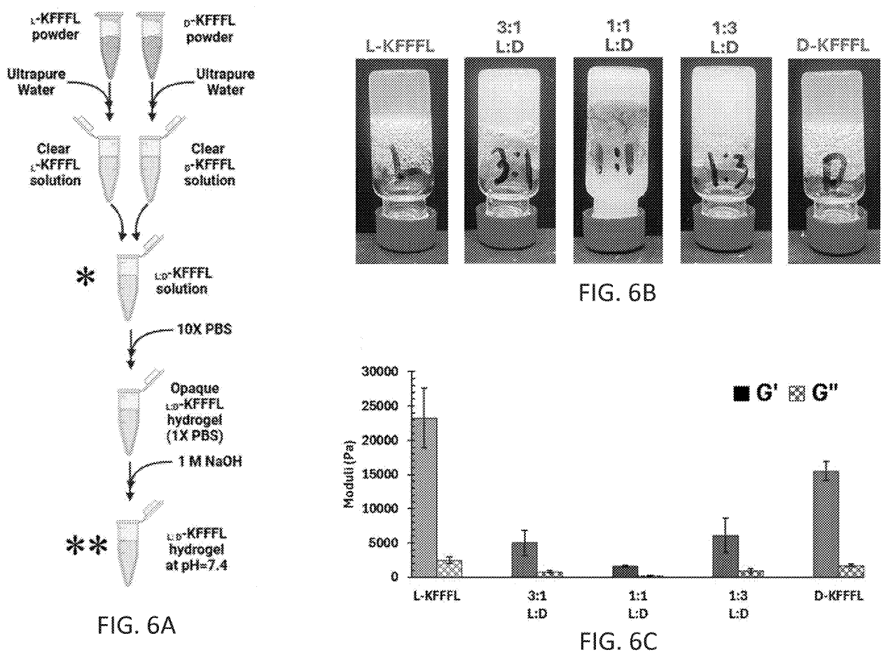

KFFFL, like KYFIL, is amphiphilic, contains an amidated C-terminus, and central phenylalanine; all sequence components necessary for RAPID hydrogel formation (FIG. 6).⁴⁸ KFFFL differs from KYFIL by the incorporation of phenylalanine (F) in place of tyrosine (Y) and isoleucine (I). Bulk properties of peptide-based biomaterials can be altered by changing as few as one amino acid from a base sequence^{48,49}. Despite the 40% change in sequence (two of five amino acids), KFFFL displays similar rheological properties to its counterpart. When completing a similar formulation process as described for KYFIL (FIG. 6A),⁴³ 1.5% (w/v) L-, 3:1 L:D-, 1:3 L:D-, and D-KFFFL readily form hydrogels within 30 minutes after salt addition and pH correction (FIG. 6B). Following the same process for 1:1 L:D-KFFFL, this mixture takes ~1-hour post-formulation for partial gelation to occur (FIG. 6B). Oscillatory shear rheology amplitude sweeps show that pure L- and D-KFFFL have the highest shear moduli, 1:1 L:D-mixtures the lowest, and 3:1 L:D- and 1:3 L:D-mixtures intermediate values (FIG. 6C). This rheological trend is consistent with the same trend for formulations with different ratios of L- and D-KYFIL,⁴³ and in FKFE (SEQ ID NO: 8) repeat motifs containing mixed versus uniform chirality⁴². Differences in bulk properties, such as storage and loss moduli, arises from stereocomplexation between L- and D-components.^{50,51} This can be in part due to stereocomplexation leading to the formation of different nanoscale morphologies.^{42-44,52}

### Impact of Salt Addition and pH Correction in Nanospiral Formation

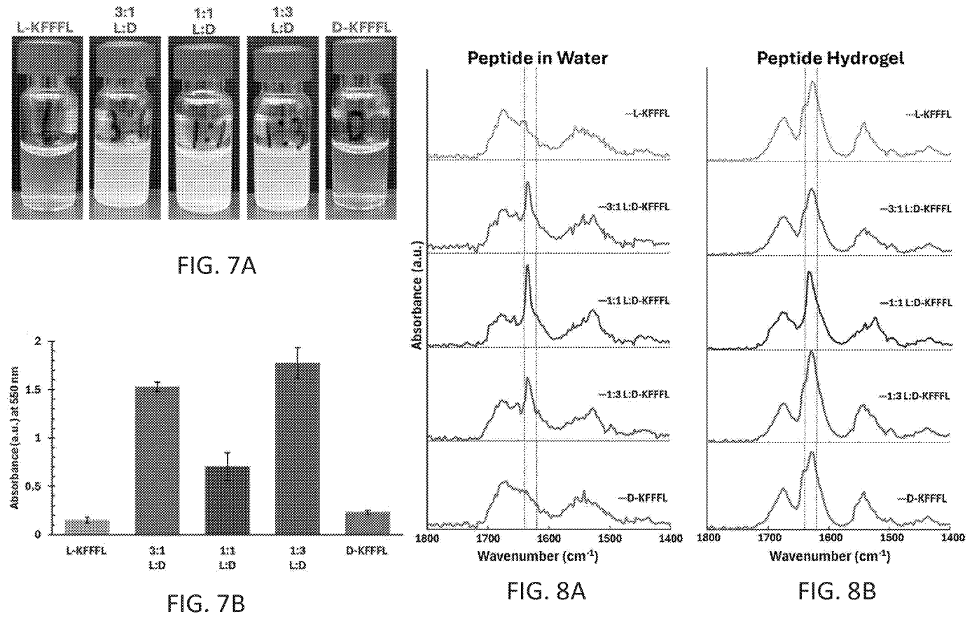

Since peptide nanospiral formation appears to be driven by stereochemistry, we next assessed the earliest signs of stereocomplexation in L:D-KFFFL mixtures. Adding ultrapure water to 1.5% (w/v) enantiomeric KFFFL mixtures results in cloudy solutions, while pure L- and D-KFFFL solubilize into clear solutions (FIGS. 7A and 7B). Although it is possible gelation may occur at a higher weight percent, as 3% (w/v) KYFIL in water forms hydrogels in 10 minutes, 43 mixtures of 1.5% (w/v) L:D-KFFFL do not form hydrogels. Pure L- and D-KFFFL contained blocky fiber-like structures, whereas L:D-KFFFL mixtures contained ribbon-like structures.

Although there is a difference in morphologies between pure L- and D-KFFFL, and L:D-KFFFL mixtures, nanospirals were not formed in any of these water formulations. Therefore, salt addition and/or pH correction impact nanospiral formation. Nanospirals form in as low as a 0.5× dilution in phosphate buffered saline.

### Secondary Structure Analysis

Next, we used Fourier transformed infrared (FTIR) spectroscopy to examine KFFFL secondary structures in solution and gain insight into nanospiral formation mechanism. In water, mixtures of (3:1 L:D-, 1:1 L:D-, and 1:3 L:D-) KFFFL reported the presence of β sheets, as indicated by a peak of ~1630 cm⁻¹ (FIG. 8A). After salt addition and pH-correction, all KFFFL formulations report β sheet formation, via a broad ~1630 cm⁻¹ peak, less sharp than those observed with peptide in water (FIG. 8B). The reported presence of β sheets compares similarly with those found in KYFIL.⁴³ Notably, KFFFL spectra contains a shoulder to the left of the ~1630 cm⁻¹ peak. This shoulder is most apparent in pure L- and D-KFFFL, slightly less noticeable in 3:1 L:D-, and 1:3 L:D-KFFFL, and vanishes in 1:1 L:D-KFFFL. These FTIR results underscore that although β sheet formation is present in nanospiral formulations, their presence alone is not sufficient for nanospiral formation. Nanospirals were not identified in any water samples, yet 3:1 L:D-, 1:1 L:D-, and 1:3 L:D-KFFFL formulations all show β sheet formation.

Nanospirals are present in 3:1 L:D-, 1:1 L:D-, and 1:3 L:D-KFFFL hydrogels, however β sheet formation is present in all hydrogel samples, including pure L- and D-KFFFL.

### Time Dependence of Nanospiral Formation

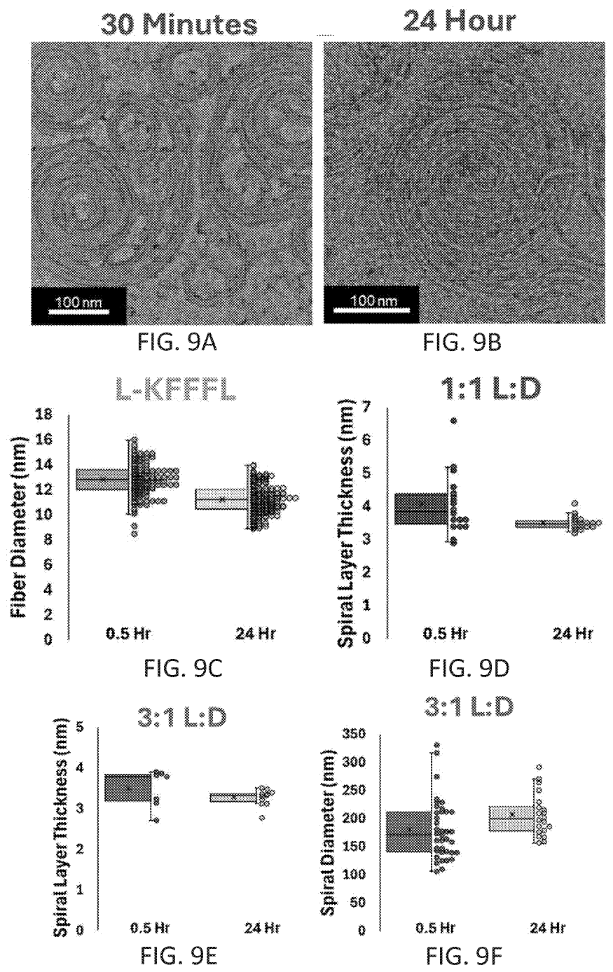

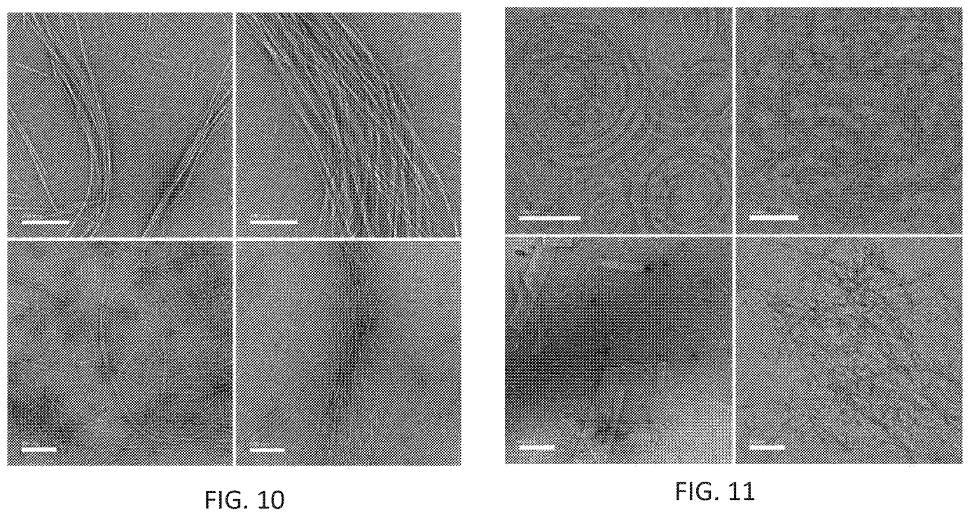

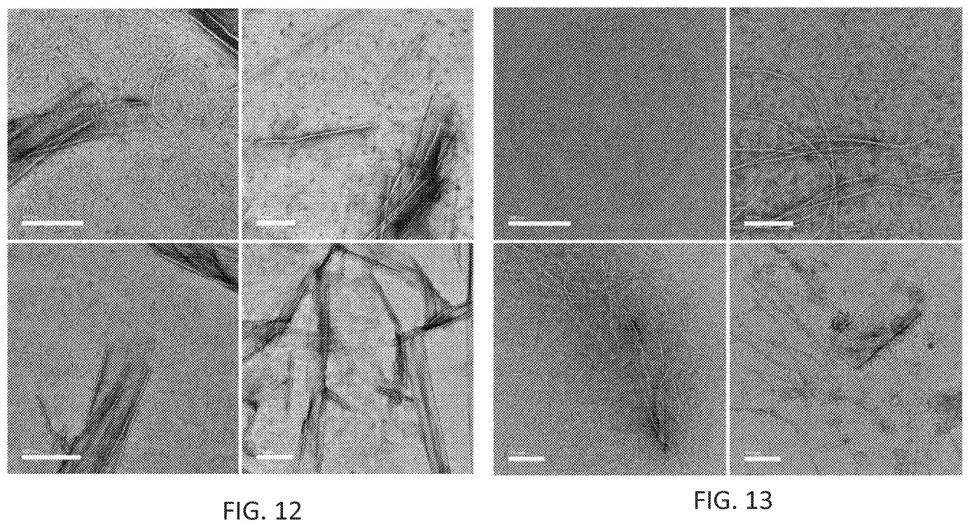

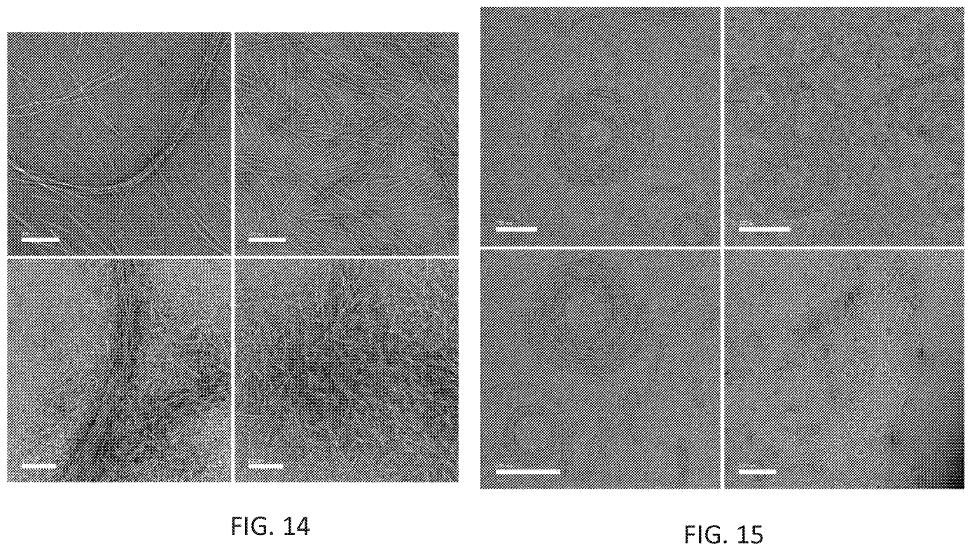

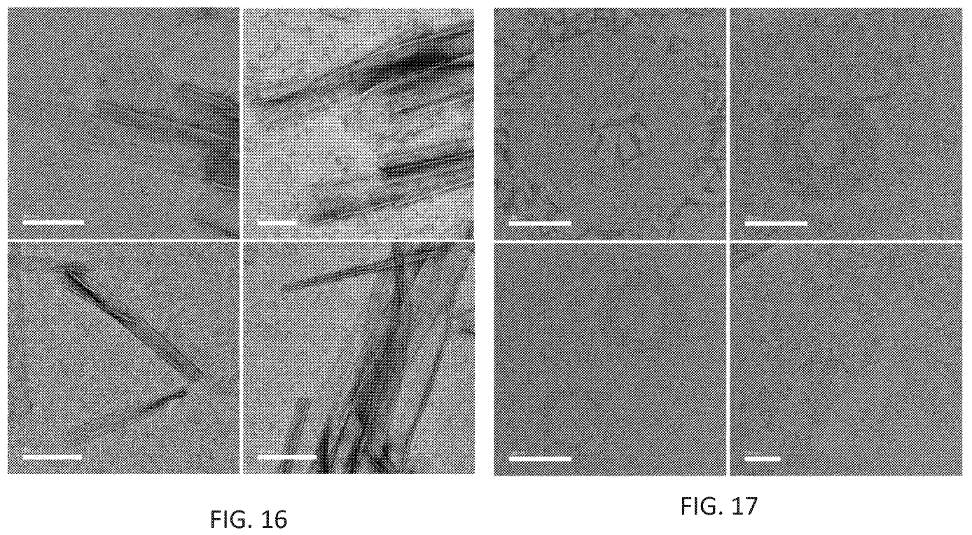

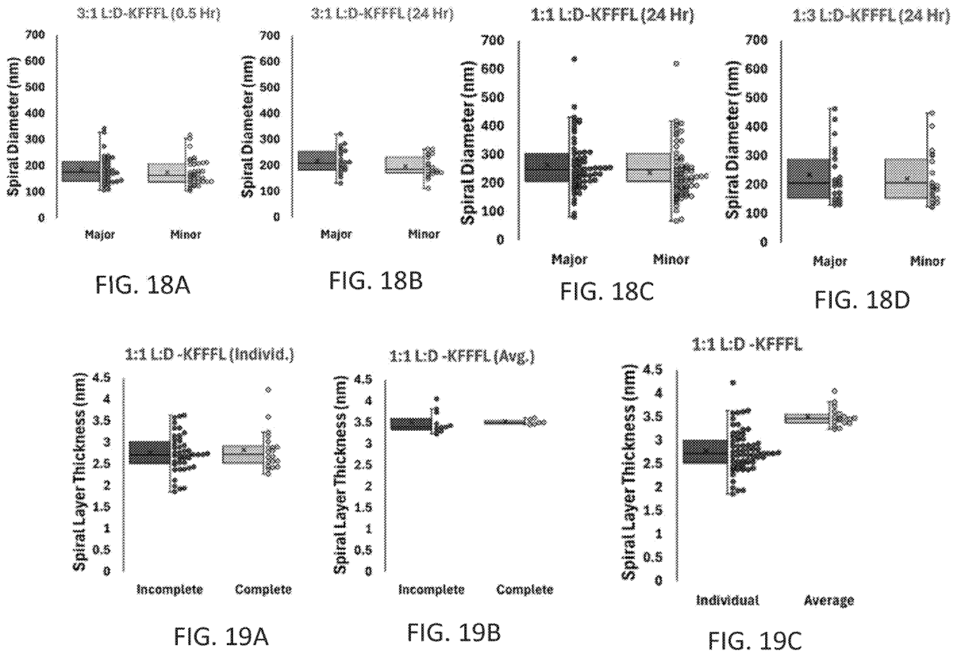

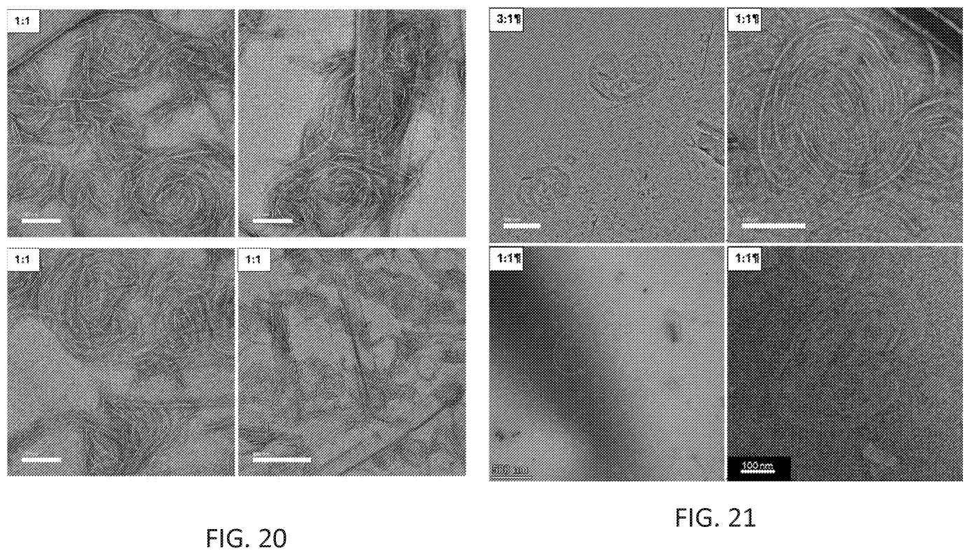

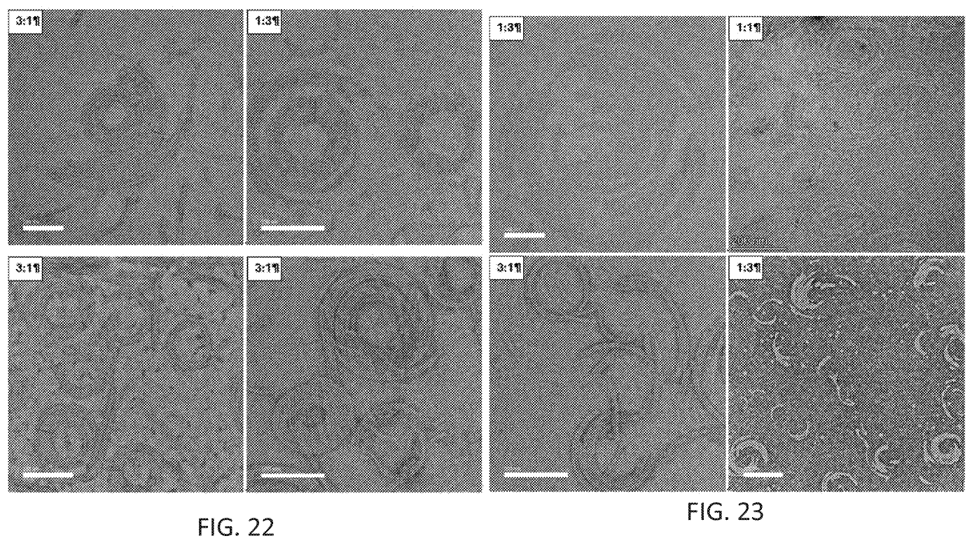

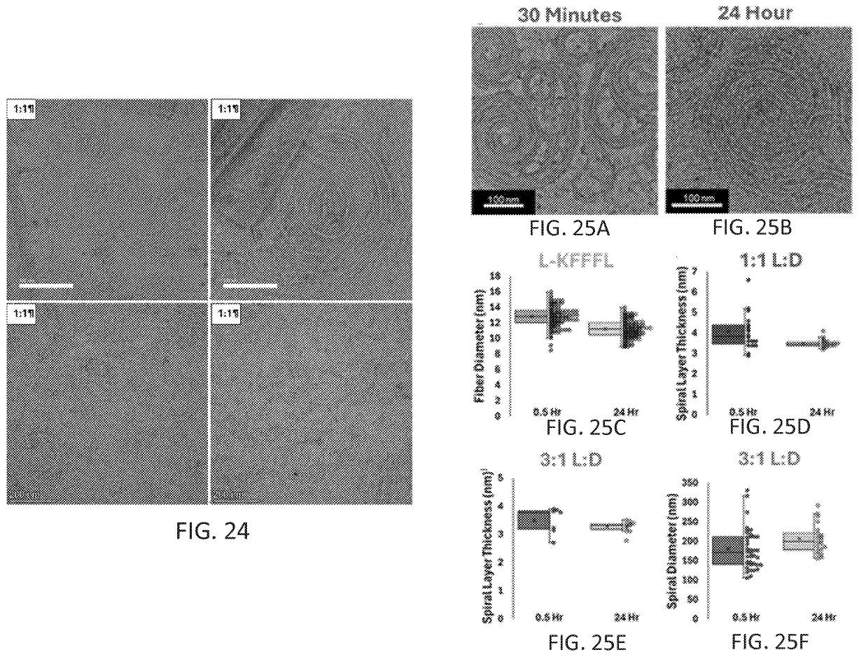

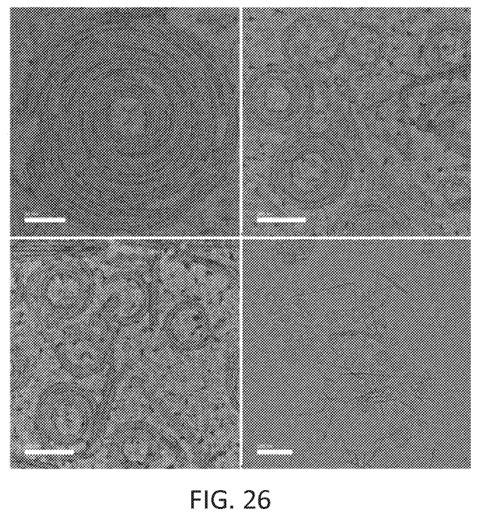

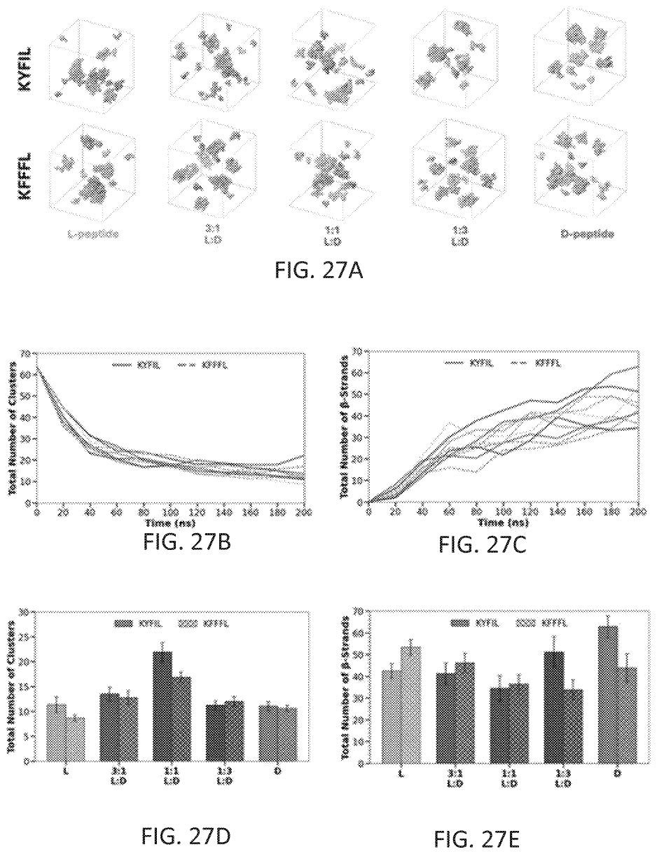

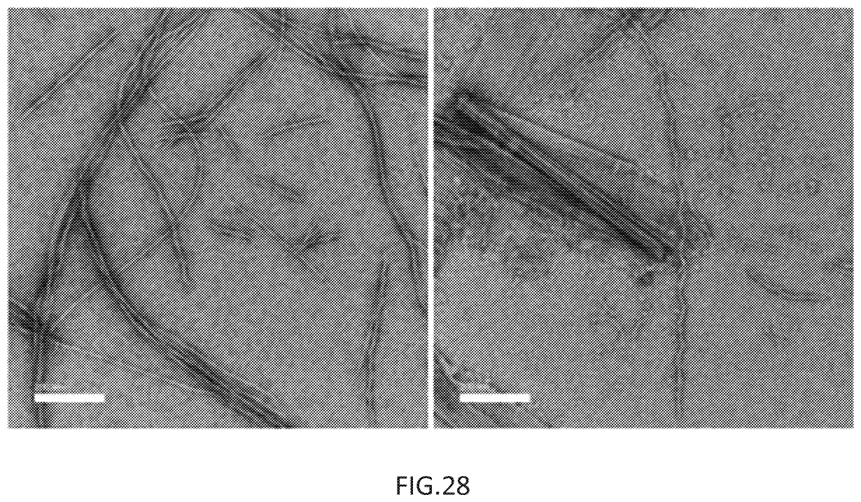

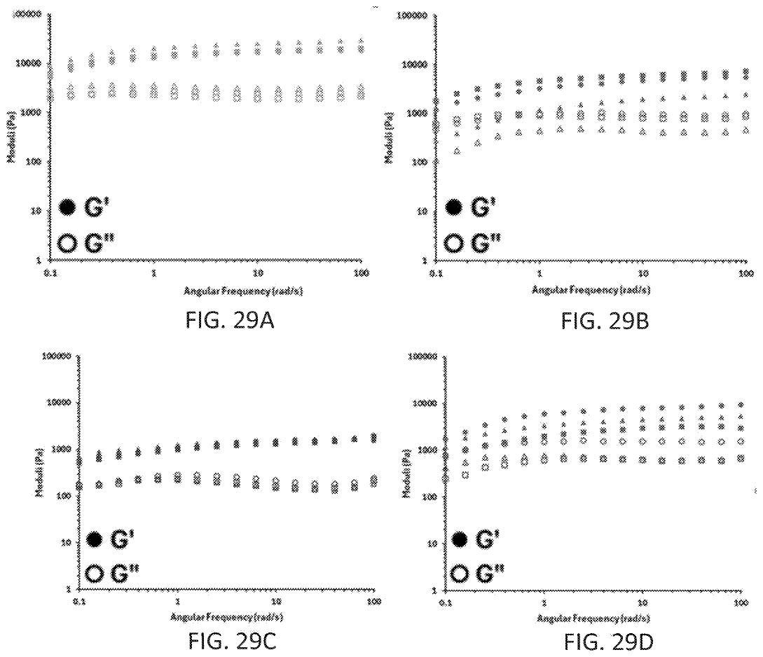

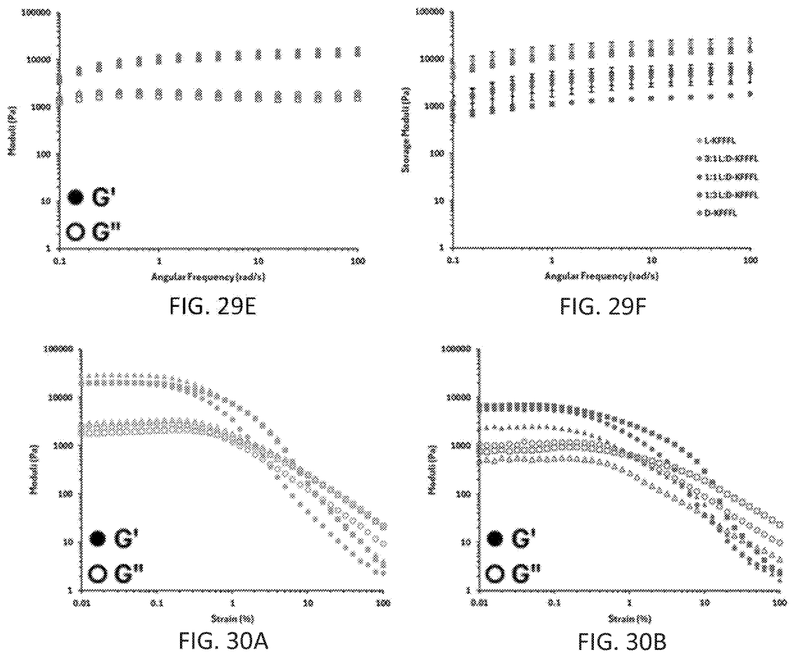

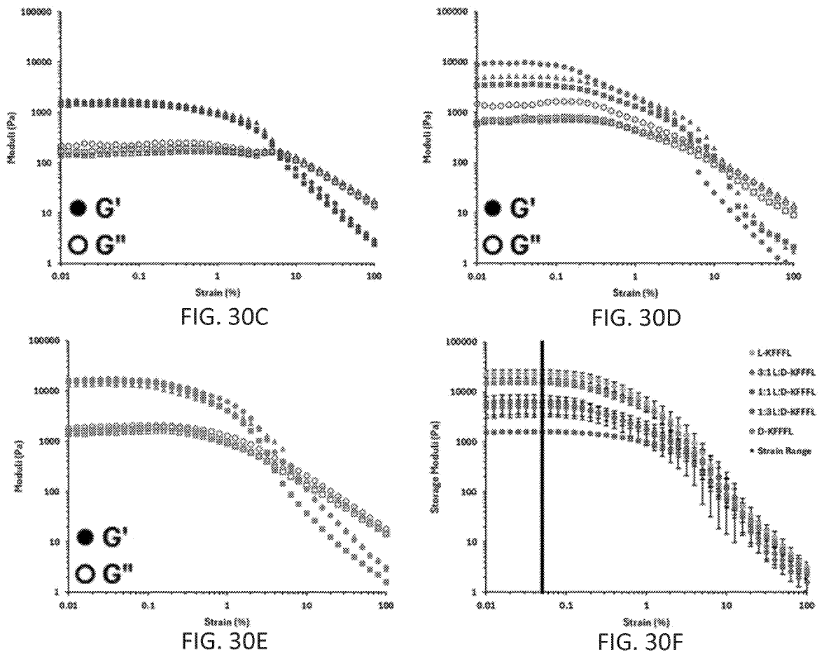

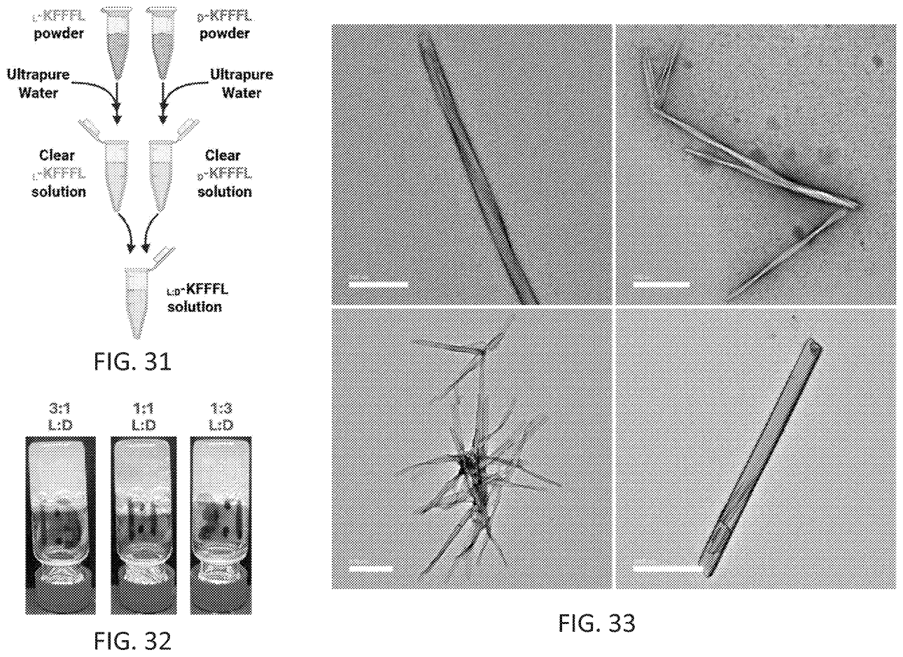

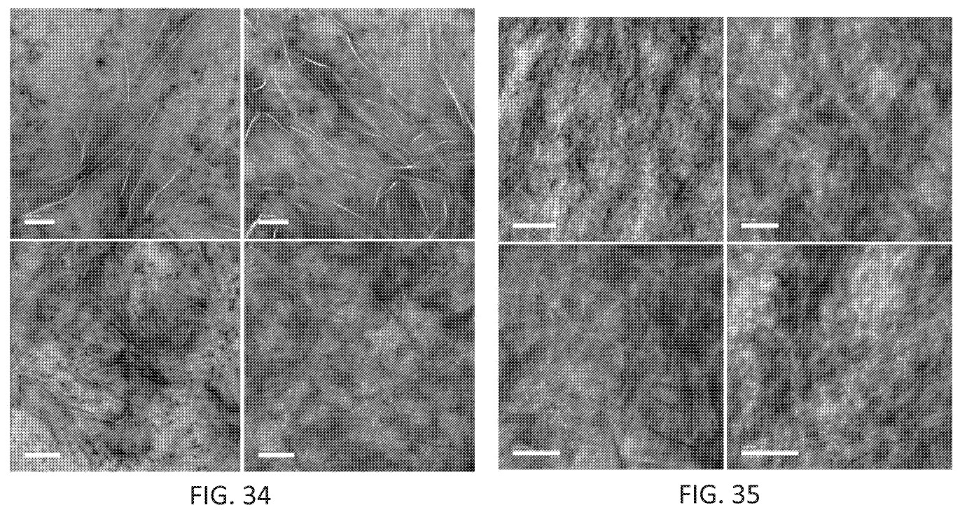

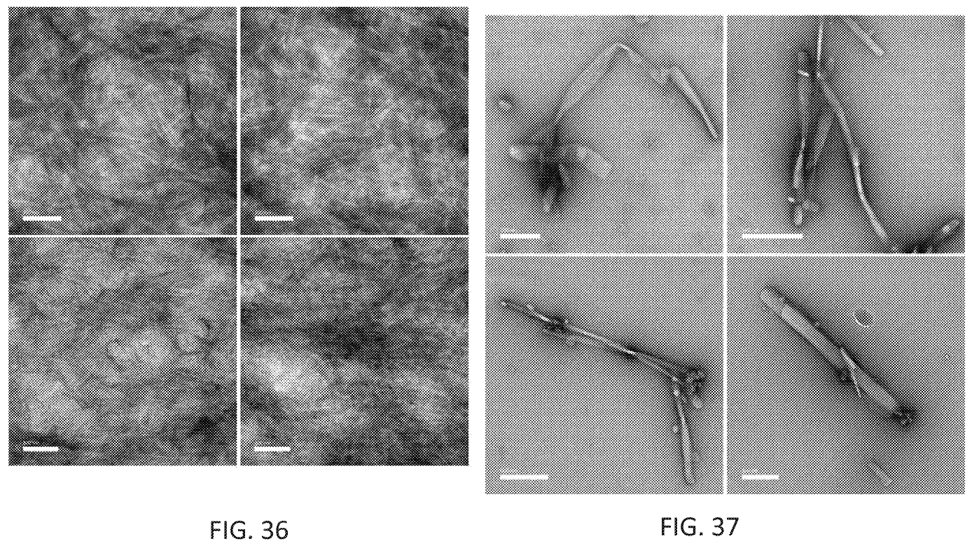

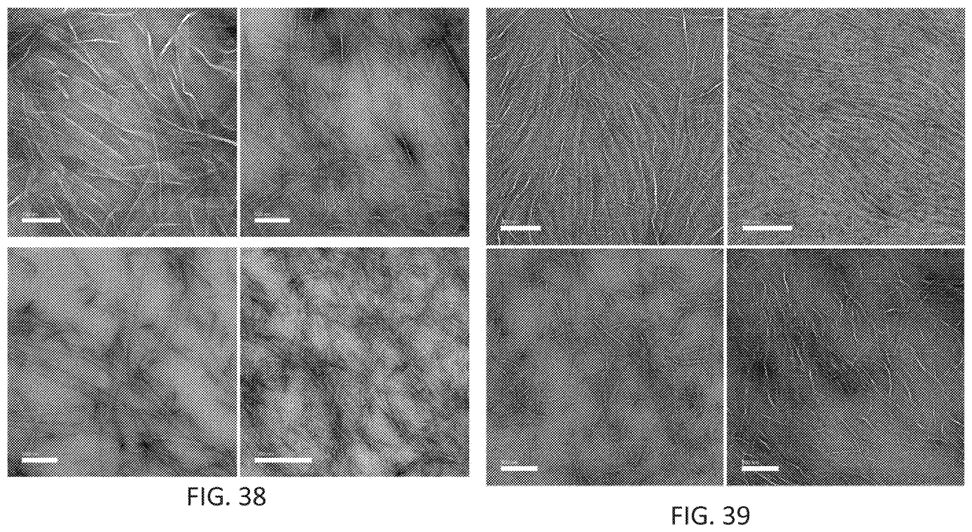

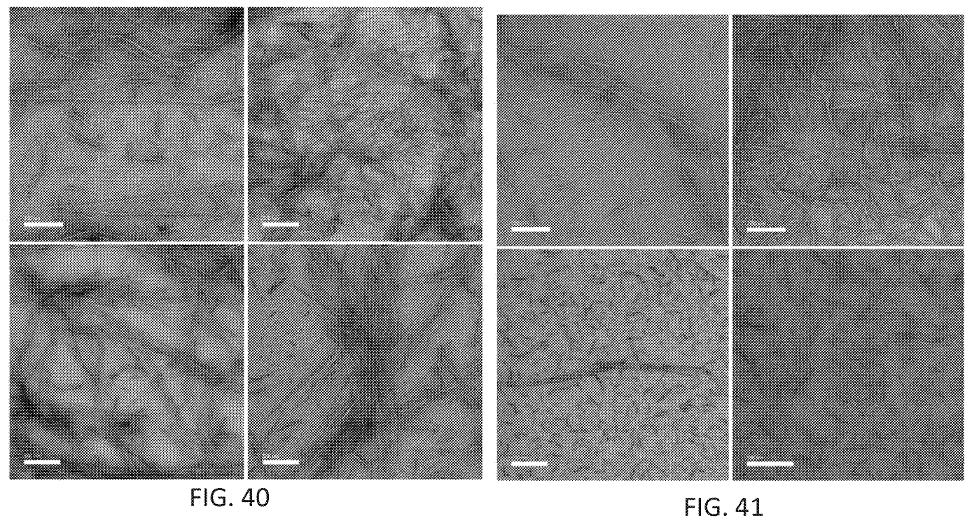

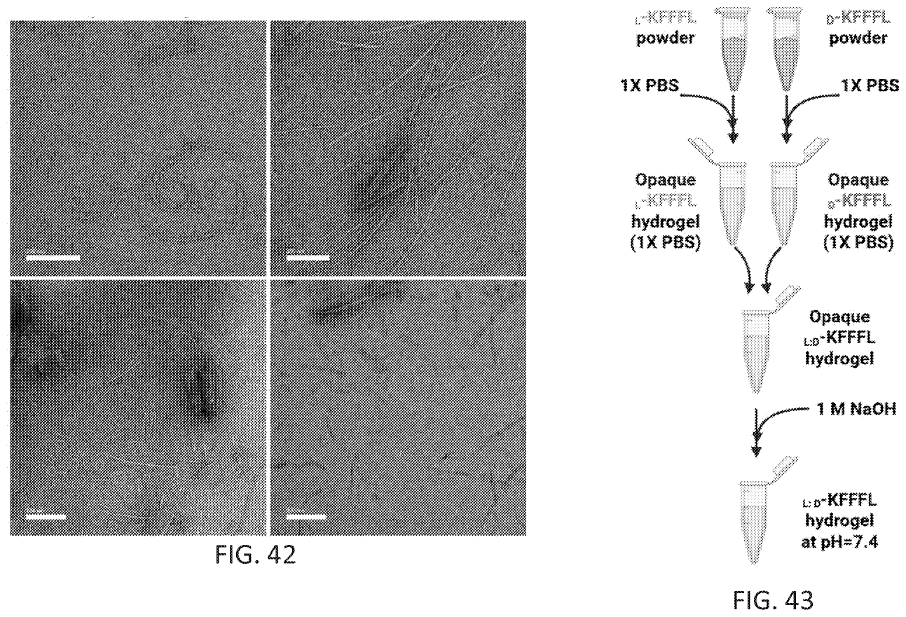

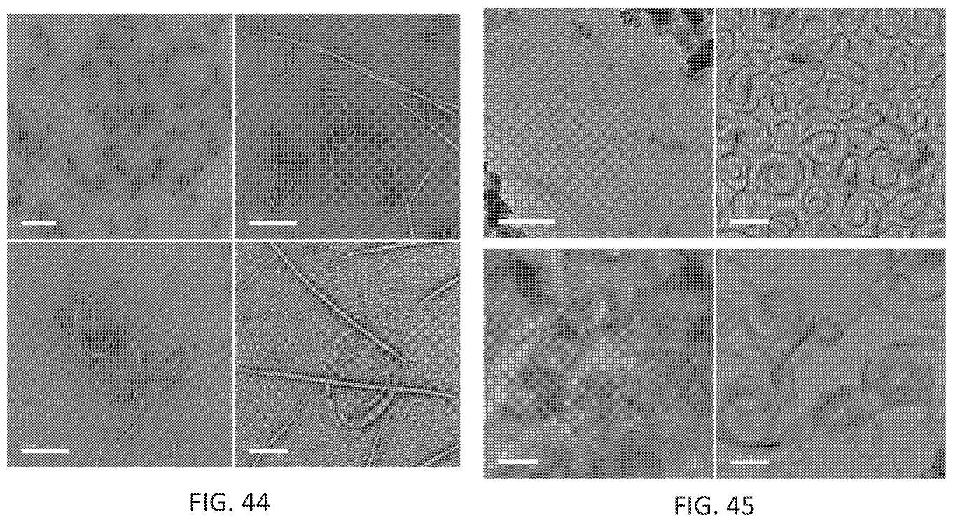

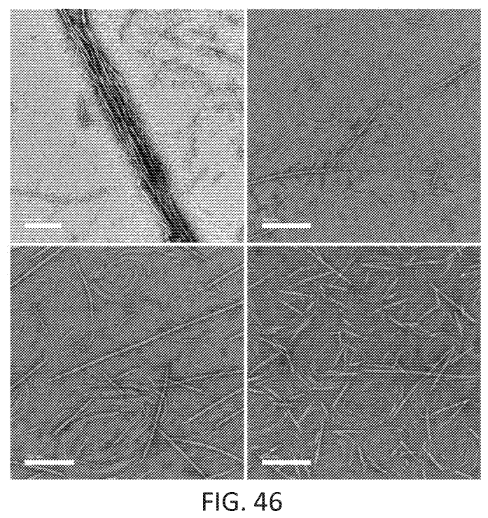

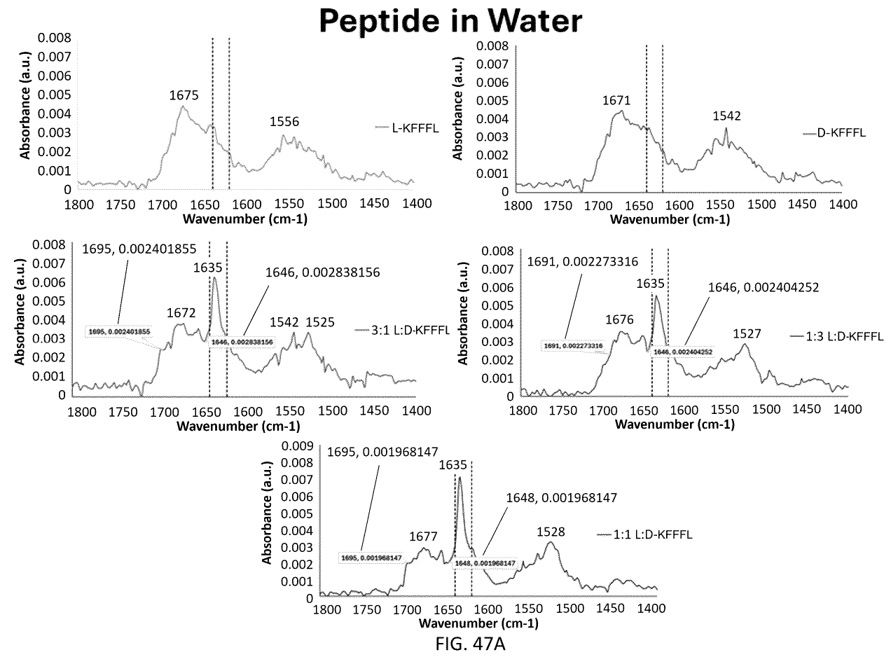

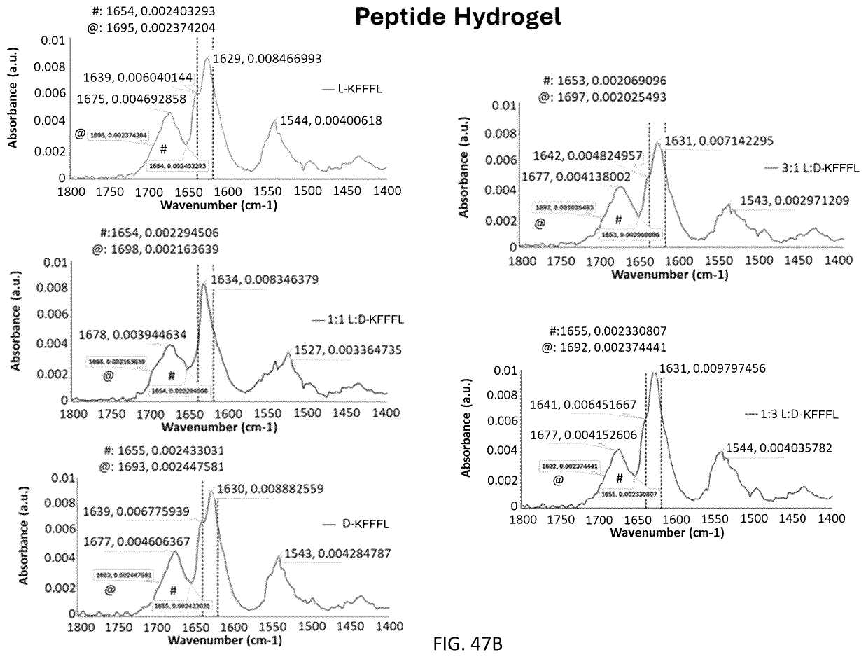

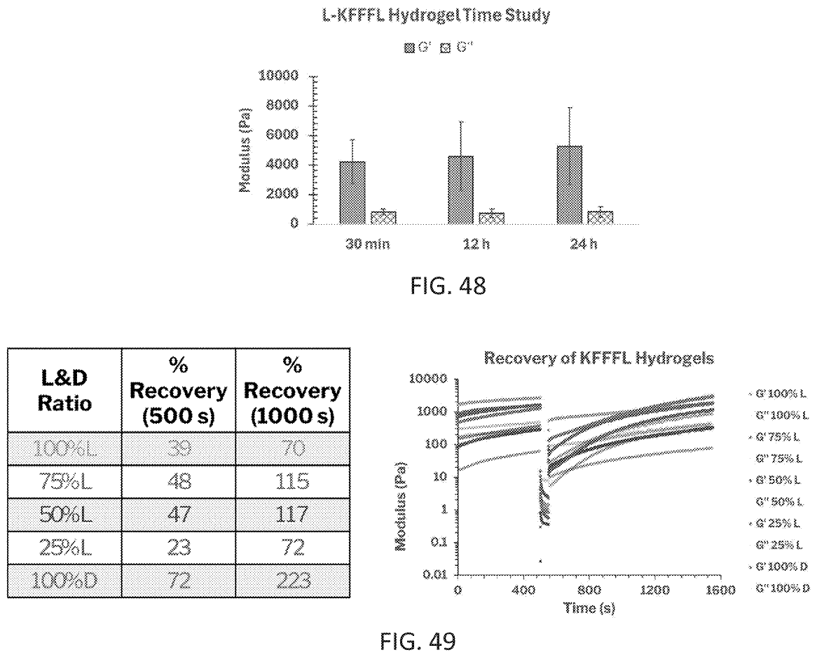

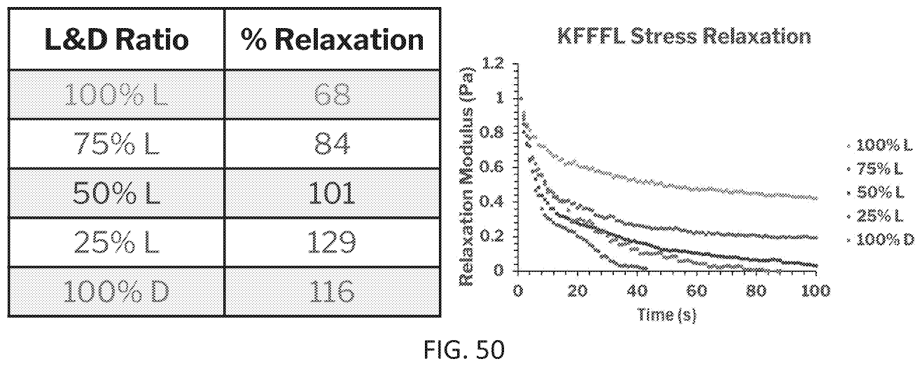

Assembly of nanostructures within self-assembling peptides can occur over various timescales,^{30,56} therefore we assessed the time dependence of nanospiral formation. In TEM images taken ~24 hours post-formulation (FIG. 9A), nanospirals appeared more complete compared to nanospirals ~30 minutes post-formulation (FIG. 9B). In both cases, spirals missing a center i.e. hollow spirals were present. However spirals at 24 hours appeared to be more packed together and regular in comparison to 30 minutes. Quantification of fiber diameter time dependence in L-KFFFL showed there was a small decrease in fiber diameter from ~30 minutes to ~24 hours (FIG. 9C). This decrease could potentially indicate more packing of fibers occurring with time. Similarly, quantification of nanospiral layer thickness in 1:1 and 3:1 L:D-KFFFL showed a small decrease, from ~30 minutes to ~24 hours (FIGS. 9D and 9E, respectively). This decrease could potentially indicate an increase in spiral layer packing with time. Quantification of nanospiral diameter time dependence in 3:1 L:D-KFFFL showed there was a small increase in nanospiral diameter from ~30 minutes to ~24 hours (FIG. 9F). This increase could potentially indicate the addition of more layers being added to spirals with time. In all cases of quantification, variance was lower in the 24 hour groups compared to 30 minute groups, suggesting spiral formation may take longer than 30 minutes to reach a thermodynamically stable structure. As previously described, taken with these results, the heterogeneity in nanospirals should be considered. Exemplary measurements of KFFL nanostructures over time are provided in Table 2.

TABLE 2
KFFFL nanostructures over time (average ± SD)

| Structure | 30 minutes | 24 hours |
|---|---|---|
| Fiber diameter | 12.8 ± 1.3 | 11.2 ± 1.2 |
| (nm; L-KFFFL) | | |
| Spiral layer thickness | 3.5 ± 0.5 | 3.3 ± 0.2 |
| (nm; 1:1 L:D) | | |
| Spiral layer thickness | 4.1 ± 0.9 | 3.5 ± 0.2 |
| (nm; 3:1 L:D) | | |
| Spiral diameter | 180 ± 53 | 207 ± 38 |
| (nm; 3:1 L:D) | | |

### CONCLUSIONS

KFFFL, a new sequence in the RAPID hydrogel family,⁴⁸ produces a spiral morphology in stereocomplexed hydrogels. Nanospirals are approximately 100-500 nm in diameter, and consist of layers 3-4 nm thick, regardless of L:D-ratio, 24 hours post-formulation. Nanospiral morphologies can be heterogeneous, appearing as rosette, incomplete, broken, or mismatched sub-morphologies. Nanospirals form after salt addition and pH correction to 7.4±0.2 in as little as 30 minutes but likely continue to assemble over longer time periods. In KFFFL hydrogels composed of different L:D ratios, nanospirals, fibers, and plate-like structures assemble via β sheet formation. Each morphology mechanism is different, with nanospirals and plate-like structures more thermodynamically favored than fibers, as they as are the only structures present in 1:1 L:D-KFFFL. Although the exact mechanism is unknown, nanospiral packing and curvature are attributed to stereocomplexation and the repeating three central phenylalanine residues.

Rheological trends for KFFFL are consistent with stereocomplexed KYFIL gels; as stereocomplexation increases, G′ and G″ decreases. However, while KFFFL stereocomplexation produces nanospirals in addition to fiber and plate-like morphologies, KYFIL produces only fiber and plate-like morphologies. This information implies nanospirals have a low impact on mechanical properties such as storage and loss modulus, although this impact is uncertain, as nanospirals only appear in the presence of other morphologies i.e. plate-like structures in 1:1 L:D-KFFFL. The discovery of a new self-assembling peptide nanostructure built from amino acids has implications on hydrogel physical properties, and biological properties.

### Materials/Methods

Hydrogel Formulation: Ultrapure water was sterilized using a polyethersulfone (PES) 0.22 μm pore size filter. L- and D-KFFFL (GenScript) were weighed out in separate Eppendorf tubes and sterilized under ultraviolet (UV) light. Each peptide was dissolved in sterile ultrapure water by stirring and pipetting up and down. Peptide solution was aliquoted into five different groups, including pure L- and D-KFFFL, and blends with volumetric ratios of 3:1, 1:1, and 1:3 L:D-KFFFL. Each enantiomeric blend was mixed by stirring with a pipette immediately after combining the previously dissolved L- and D-components. The formulation process for peptide in water stopped at FIG. 6*. Hydrogel formulation continued by adding sterile-filtered 10× phosphate buffered saline (PBS) to the peptide solutions to create a final concentration of 1×PBS. Sterile-filtered 1M NaOH was added until each sample reached a pH of 7.4±0.2 (FIG. 6**).

Oscillatory Shear Rheology: Rheological measurements were conducted on an Anton Paar Modular Compact Rheometer 302e. All tests were completed at room temperature using a 25 mm diameter parallel plate geometry with a gap height of 500 μm on peptide concentrations of 1.5% (w/v) L-, 3:1 L:D-, 1:1 L:D-, 1:3 L:D-, and D-KFFFL (FIG. 6C). Frequency sweeps were performed from 0.1-100 rad/s at a constant strain of 0.1%. Directly following, amplitude sweeps were performed with a constant angular frequency of 10 rad/s from 0.01-101% strain. Storage and loss moduli shown are averaged values within the linear viscoelastic range of 0.01-0.05% strain. Rheology was completed for N=3 independently prepared samples for each experimental group (FIG. 6C).

### Transmission Electron Microscopy:

Imaging: Samples (5 μL) prepared at 1.5% (w/v), 0.75% (w/v), and 0.5% (w/v) in water, and 1.5% (w/v) after salt addition and pH correction (FIGS. 3, 4, 5, 6, and 9) were applied onto glow-discharged carbon film grids. Excess solvent was removed via blotting. For elastic samples, excess hydrogel was trimmed by carefully sliding a razor blade along the grid surface. Grids were lowered into 20 μl 2% (w/v) uranyl acetate stain droplets for 1 minute before blotting to dryness. Images were collected on an FEI Titan transmission electron microscope (Thermo-Fisher) operating at 120 kV. One set of images were collected using an FEI Themis Z transmission electron microscope (Thermo-Fisher) operating at 80 kV. Spiral measurements were collected from N=3 hydrogels per experimental group with all 24-hour images taken on the FEI Titan transmission electron microscope (FIGS. 3, 4, 5, 6, 9A, 9B, 9E, and 9F), and one sample of 0.5-hour images taken on the FEI Themis Z microscope (FIGS. 9C and 9D).

Quantification: After imaging was completed, the entire set of images was scanned for quantifiable nanostructures. Quality images of nanospirals were identified with the following requirements: for major and minor axis measurements, only complete spirals with a singular origin were selected. These spirals could be partially hollow (ideally less than 30% hollow) and partially unwound spirals if the unwound “tail” was not a significant portion of the full spiral. The following spirals were not quantified: spirals that were merged or superimposed onto other spirals, spirals with unclear axis borders, spirals made up of multiple separate fragments, spirals that were jagged spirals i.e. rosettes, spirals with undiscernible intra-fiber spacings. If an otherwise complete spiral had a fragment attached to it, the fragment portion was not measured. For measurements of nanofibers within spirals, areas with uniform fiber spacing were selected, and could include incomplete spirals or spiral fragments.

Simulation Procedure: Simulations were carried out over 200 ns with 64 peptides (3 wt %) of KYFIL or KFFFL using a Modified CHARMM force field and NAMD simulation software. Each simulation was equilibrated for 1 ns, wherein NaCl was added to neutralize the system and achieve a physiological salt concentration of 150 mM. Five peptide compositions of L-, 3:1 L:D-, 1:1 L:D-, 1:3 L:D-, and D-peptide were investigated (FIG. 4). Raw data is provided in Table 3.

TABLE 3
Simulation raw data for last 20 ns (avg. ± SD)

| | Hydrogen Bonding | | Clustering | | β-Strands | | π-π Stacking | |
|---|---|---|---|---|---|---|---|---|
| | KYFIL | KFFFL | KYFIL | KFFFL | KYFIL | KFFFL | KYFIL | KFFFL |
| L-peptide | 50.7 ± 4.3 | 53.2 ± 5.1 | 11.3 ± 1.6 | 8.70 ± 0.64 | 42.5 ± 3.5 | 53.3 ± 3.6 | 5.89 ± 2.2 | 22.4 ± 4.2 |
| 3:1 L:D | 47.2 ± 4.5 | 45.3 ± 4.5 | 13.5 ± 1.4 | 12.8 ± 1.4 | 41.3 ± 5.0 | 46.2 ± 4.4 | 10.1 ± 2.5 | 18.4 ± 3.7 |
| 1:1 L:D | 28.3 ± 3.7 | 34.3 ± 3.7 | 21.9 ± 2.0 | 16.9 ± 1.0 | 34.5 ± 5.9 | 36.5 ± 4.4 | 5.40 ± 2.2 | 14.9 ± 3.5 |
| 1:3 L:D | 40.4 ± 4.1 | 38.4 ± 4.5 | 11.2 ± 0.88 | 12.0 ± 1.0 | 51.3 ± 7.0 | 33.9 ± 4.6 | 6.71 ± 2.2 | 19.0 ± 3.7 |
| D-peptide | 43.1 ± 4.5 | 43.8 ± 4.5 | 11.0 ± 1.0 | 10.6 ± 0.65 | 62.8 ± 5.1 | 43.9 ± 6.4 | 9.77 ± 2.5 | 18.9 ± 3.6 |

Absorbance Measurements: Formulations of 1.5% (w/v) L-, 3:1 L:D-, 1:1 L:D-, 1:3 L:D-, and D-KFFFL in sterile ultrapure water (FIG. 4A) were placed in a UV-STAR® 96-well microplate 30 minutes after peptide was fully dissolved and mixed. Absorbance measurements were acquired at 550 nm using a BMG LABTECH CLARIOstar®
Fourier Transformed Infrared Spectroscopy: FTIR spectra was obtained using a PerkinElmer 400 FTIR spectrometer equipped with an attenuated total reflectance accessory at 1 cm⁻¹ resolution. Spectra in FIG. 8A is for 1.5% (w/v) L-, 3:1 L:D-, 1:1 L:D-, 1:3 L:D-, and D-KFFFL in sterile ultrapure water. Spectra in FIG. 8B is for fully formulated 3% (w/v) L-, 3:1 L:D-, 1:1 L:D-, 1:3 L:D-, and D-KFFFL. FTIR analysis of KFFFL hydrogels was performed at a 3% (w/v) for greater consistency and confidence in measurements.

### REFERENCES

1. Zhang, S. Discovery and Design of Self-Assembling Peptides. Interface Focus 2017, 7 (6), 20170028. doi.org/10.1098/rsfs.2017.0028.
2. Yamada, Y.; Patel, N. L.; Kalen, J. D.; Schneider, J. P. Design of a Peptide-Based Electronegative Hydrogel for the Direct Encapsulation, 3D Culturing, in Vivo Syringe-Based Delivery, and Long-Term Tissue Engraftment of Cells. ACS Appl. Mater. Interfaces 2019, 11 (38), 34688-34697. doi.org/10.1021/acsami.9b12152.
3. Nguyen, A. K. et al. Hierarchical Assembly of Tryptophan Zipper Peptides into Stress-Relaxing Bioactive Hydrogels. Nat Commun 2023, 14 (1), 6604. doi.org/10.1038/s41467-023-41907-1.
4. Sleep, E. et al. Injectable Biomimetic Liquid Crystalline Scaffolds Enhance Muscle Stem Cell Transplantation. Proceedings of the National Academy of Sciences 2017, 114 (38), E7919-E7928. doi.org/10.1073/pnas. 1708142114.
5. Nayak, S. et al. Cystine-Cored Diphenylalanine Appended Peptide-Based Self-Assembled Fluorescent Nanostructures Direct Redox-Responsive Drug Delivery. iScience 2024, 27 (4), 109523. doi.org/10.1016/j.isci.2024.109523.
6. Guo, S.; Wang, J.; Wang, Q.; Wang, J.; Qin, S.; Li, W. Advances in Peptide-Based Drug Delivery Systems. Heliyon 2024, 10 (4), e26009. doi.org/10.1016/j.heliyon.2024.e26009.
7. Al-azzawi, S. et al. Dendrimeric Poly(Epsilon-Lysine) Delivery Systems for the Enhanced Permeability of Flurbiprofen across the Blood-Brain Barrier in Alzheimer's Disease. International Journal of Molecular Sciences 2018, 19 (10), 3224. doi.org/10.3390/ijms19103224.
8. Guo, J.; Su, H.; Zeng, Y.; Liang, Y.-X.; Wong, W. M.; Ellis-Behnke, R. G.; So, K.-F.; Wu, W. Reknitting the Injured Spinal Cord by Self-Assembling Peptide Nanofiber Scaffold. Nanomedicine 2007, 3 (4), 311-321. doi.org/10.1016/j.nano.2007.09.003.
9. Chen, C. H. et al. Supramolecular Self-Assembling Peptides to Deliver Bone Morphogenetic Proteins for Skeletal Regeneration. Bone 2020, 141, 115565. doi.org/10.1016/j.bone.2020.115565.
10. Li, S.; Yu, Q.; Li, H.; Chen, M.; Jin, Y.; Liu, D. Self-Assembled Peptide Hydrogels in Regenerative Medicine. Gels 2023, 9 (8), 653. doi.org/10.3390/gels9080653.
11. Wang, J.; Liu, K.; Xing, R.; Yan, X. Peptide Self-Assembly: Thermodynamics and Kinetics. Chem. Soc. Rev. 2016, 45 (20), 5589-5604. doi.org/10.1039/C6CS00176A.
12. Edwards-Gayle, C. J. C.; Hamley, I. W. Self-Assembly of Bioactive Peptides, Peptide Conjugates, and Peptide Mimetic Materials. Org. Biomol. Chem. 2017, 15 (28), 5867-5876. doi.org/10.1039/C7OB01092C.
13. Wang, Y.; Qi, W.; Huang, R.; Yang, X.; Wang, M.; Su, R.; He, Z. Rational Design of Chiral Nanostructures from Self-Assembly of a Ferrocene-Modified Dipeptide. J. Am. Chem. Soc. 2015, 137 (24), 7869-7880. doi.org/10.1021/jacs.5b03925.
14. Hutchinson, J. A. et al. Peptide Hormones and Lipopeptides: From Self-assembly to Therapeutic Applications. J Pept Sci 2017, 23 (2), 82-94. doi.org/10.1002/psc.2954.
15. Gray, V. P.; Amelung, C. D.; Duti, I. J.; Laudermilch, E. G.; Letteri, R. A.; Lampe, K. J. Biomaterials via Peptide Assembly: Design, Characterization, and Application in Tissue Engineering. Acta Biomaterialia 2022, 140, 43-75. doi.org/10.1016/j.actbio.2021.10.030.
16. Branco, M. C.; Schneider, J. P. Self-Assembling Materials for Therapeutic Delivery. Acta biomaterialia 2008, 5 (3), 817. doi.org/10.1016/j.actbio.2008.09.018.
17. Wang, Y et al. Self-Assembled Peptide with Morphological Structure for Bioapplication. Biomacromolecules 2024, 25 (10), 6367-6394. doi.org/10.1021/acs.biomac.4c01179.
18. Cook, S. T. A.; Cook, T. A. The Curves of Life: Being an Account of Spiral Formations and Their Application to Growth in Nature, to Science, and to Art: With Special Reference to the Manuscripts of Leonardo Da Vinci; Courier Corporation, 1979.
19. Choi, H. K. et al. Nanoscale Spirals by Directed Self-Assembly. Nano Futures 2017, 1 (1), 015001. doi.org/10.1088/2399-1984/aa641c.
20. Chai, J.; Buriak, J. M. Using Cylindrical Domains of Block Copolymers To Self-Assemble and Align Metallic Nanowires. ACS Nano 2008, 2 (3), 489-501. doi.org/10.1021/nn700341s.
21. Yagai, S. et al. Supramolecular Polymers Capable of Controlling Their Topology. Acc. Chem. Res. 2019, 52 (5), 1325-1335. doi.org/10.1021/acs.accounts.8b00660.
22. Li, H.; Liu, Q.; Qin, L.; Xu, M.; Lin, X.; Yin, S.; Wu, L.; Su, Z.; Shen, J. Self-Assembling Structures and Thin-Film Microscopic Morphologies of Amphiphilic Rod-Coil Block Oligomers. J Colloid Interface Sci 2005, 289 (2), 488-497. doi.org/10.1016/j.jcis.2005.03.052.
23. Hong, D.-J.; Lee, E.; Choi, M.-G.; Lee, M. Self-Organized Spiral Columns in Laterally Grafted Rods. Chem. Commun. 2010, 46 (27), 4896-4898. doi.org/10.1039/C0CC00132E.
24. Sasaki, N. et al. Supramolecular Double-Stranded Archimedean Spirals and Concentric Toroids. Nat Commun 2020, 11 (1), 3578. doi.org/10.1038/s41467-020-17356-5.
25. Gopal, A.; Varghese, R.; Ajayaghosh, A. Oligo (p-Phenylene-Ethynylene)-Derived Super-π-Gelators with Tunable Emission and Self-Assembled Polymorphic Structures. Chemistry—An Asian Journal 2012, 7 (9), 2061-2067. doi.org/10.1002/asia.201200410.
26. Huang, X. et al. Self-Assembled Spiral Nanoarchitecture and Supramolecular Chirality in Langmuir Blodgett Films of an Achiral Amphiphilic Barbituric Acid. J. Am. Chem. Soc. 2004, 126 (5), 1322-1323. doi.org/10.1021/ja036878i.
27. Huang, X. et al. Supramolecular Chirality of the Hydrogen-Bonded Complex Langmuir-Blodgett Film of Achiral Barbituric Acid and Melamine. Journal of Colloid and Interface Science 2005, 285 (2), 680-685. doi.org/10.1016/j.jcis.2004.12.025.
28. Zhang, Y. et al. Controllable Fabrication of Supramolecular Nanocoils and Nanoribbons and Their Morphology-Dependent Photoswitching. J. Am. Chem. Soc. 2009, 131 (8), 2756-2757. doi.org/10.1021/ja805891k.
29. Alvarez-Fernandez, A. et al. Peptide-Guided Self-Assembly: Fabrication of Tailored Spiral-Like Nanostructures for Precise Inorganic Templating. Advanced Functional Materials 2025, 35 (1), 2411061. doi.org/10.1002/adfm.202411061.
30. Wen, H. et al. Silk-Derived Peptide Nanospirals Assembled by Self-Propelled Worm-like Filaments. Nano Res. 2023, 16 (1), 1414-1420. doi.org/10.1007/s12274-022-4671-1.
31. Wang, Y. et al. Self-Assembly of Fibonacci Number Spirals in Amyloid-like Nanofibril Films. Sci. China Mater. 2022, 65 (11), 3150-3156. doi.org/10.1007/s40843-022-2087-3.
32. Hübner, R. Golden Spiral or Fibonacci Spiral: Which Is More Beautiful and Why? i-Perception 2024. doi.org/10.1177/20416695241243319.
33. Liu, J.; Zhang, J.; Zhang, L.; Wang, Y.; Wei, H.; Shen, Y.; Min, J.; Rong, X.; Qi, W.; Su, R.; He, Z. Hierarchical Metal-Peptide Assemblies with Chirality-Encoded Spiral Architecture and Catalytic Activity. Sci. China Chem. 2023, 66 (1), 228-241. doi.org/10.1007/s11426-022-1351-1.
34. Reches, M.; Gazit, E. Self-Assembly of Peptide Nanotubes and Amyloid-like Structures by Charged-Termini-Capped Diphenylalanine Peptide Analogues. Israel Journal of Chemistry 2005, 45 (3), 363-371. doi.org/10.1560/5MC0-V3DX-KEOB-YF3J.
35. Reches, M.; Gazit, E. Casting Metal Nanowires Within Discrete Self-Assembled Peptide Nanotubes. Science 2003, 300 (5619), 625-627. doi.org/10.1126/science.1082387.
36. Dutta, S. et al. Suppression of Oligomer Formation and Formation of Non-Toxic Fibrils upon Addition of Mirror-Image Aβ42 to the Natural 1-Enantiomer. Angewandte Chemie International Edition 2017, 56 (38), 11506-11510. doi.org/10.1002/anie.201706279.
37. Urban, J. M.; Ho, J.; Piester, G.; Fu, R.; Nilsson, B. L. Rippled β-Sheet Formation by an Amyloid-β Fragment Indicates Expanded Scope of Sequence Space for Enantiomeric β-Sheet Peptide Coassembly. Molecules 2019, 24 (10), 1983. doi.org/10.3390/molecules24101983.
38. Tamamis, P. et al. Self-Assembly of Phenylalanine Oligopeptides: Insights from Experiments and Simulations. Biophys J 2009, 96 (12), 5020-5029. doi.org/10.1016/j.bpj.2009.03.026.
39. Mayans, E. et al. Diversity and Hierarchy in Supramolecular Assemblies of Triphenylalanine: From Laminated Helical Ribbons to Toroids. Langmuir 2017, 33 (16), 4036-4048. doi.org/10.1021/acs.langmuir.7b00622.
40. Mosseri, A. et al. Chiral Fibers Formation Upon Assembly of Tetraphenylalanine Peptide Conjugated to a PNA Dimer. Chemistry—A European Journal 2022, 28 (37), e202200693. doi.org/10.1002/chem.202200693.
41. Mayans, E. et al. Self-Assembly of Tetraphenylalanine Peptides. Chemistry—A European Journal 2015, 21 (47), 16895-16905. doi.org/10.1002/chem.201501793.
42. Clover, T. M. et al. Self-Assembly of Block Heterochiral Peptides into Helical Tapes. J. Am. Chem. Soc. 2020, 142 (47), 19809-19813. doi.org/10.1021/jacs.9b09755.
43. Duti, I. J.; Florian, J. R.; Kittel, A. R.; Amelung, C. D.; Gray, V. P.; Lampe, K. J.; Letteri, R. A. Peptide Stereocomplexation Orchestrates Supramolecular Assembly of Hydrogel Biomaterials. J. Am. Chem. Soc. 2023, 145 (33), 18468-18476. doi.org/10.1021/jacs.3c04872.
44. Jeena, M. T. et al. Heterochiral Assembly of Amphiphilic Peptides Inside the Mitochondria for Supramolecular Cancer Therapeutics. ACS Nano 2019, 13 (10), 11022-11033. doi.org/10.1021/acsnano.9b02522.
45. Gray, V. P.; Letteri, R. A. Designing Coiled Coils for Heterochiral Complexation to Enhance Binding and Enzymatic Stability. Biomacromolecules 2024, 25 (8), 5273-5280. doi.org/10.1021/acs.biomac.4c00661.
46. Bomb, K.; Zhang, Q.; Ford, E. M.; Fromen, C. A.; Kloxin, A. M. Systematic D-Amino Acid Substitutions to Control Peptide and Hydrogel Degradation in Cellular Microenvironments. ACS Macro Lett. 2023, 12 (6), 725-732. doi.org/10.1021/acsmacrolett.3c00144.
47. Griffin, D. R. et al. Activating an Adaptive Immune Response from a Hydrogel Scaffold Imparts Regenerative Wound Healing. Nat. Mater. 2021, 20 (4), 560-569. doi.org/10.1038/s41563-020-00844-w.
48. Tang, J. D. et al. Stimuli-Responsive, Pentapeptide, Nanofiber Hydrogel for Tissue Engineering. J. Am. Chem. Soc. 2019, 141 (12), 4886-4899. doi.org/10.1021/jacs.8b13363.
49. Dooling, L. J.; Tirrell, D. A. Engineering the Dynamic Properties of Protein Networks through Sequence Variation. ACS Cent. Sci. 2 2016, (11), 812-819. doi.org/10.1021/acscentsci.6b00205.
50. Swanekamp, R. J.; Welch, J. J.; Nilsson, B. L. Proteolytic Stability of Amphipathic Peptide Hydrogels Composed of Self-Assembled Pleated β-Sheet or Coassembled Rippled β-Sheet Fibrils. Chem. Commun. 2014, 50 (70), 10133-10136. doi.org/10.1039/C4CC04644G.
51. Ahmed, J. et al. Rheological and Thermal Properties of Stereocomplexed Polylactide Films. J Therm Anal calorim 2014, 115 (3), 2053-2061. doi.org/10.1007/s10973-013-3234-9.
52. Xie, Y.; Yu, W.; Xia, T.; O'Reilly, R. K.; Dove, A. P. Stereocomplex-Driven Morphological Transition of Coil-Rod-Coil Poly(Lactic Acid)-Based Cylindrical Nanoparticles. Macromolecules 2023, 56 (19), 7689-7697. doi.org/10.1021/acs.macromol.3c00653.
53. Size of amino acid molecule estimated based o—Generic—BNID 106983. bionumbers.hms.harvard.edu/bionumber.aspx?&id=106983 (accessed 2025 Mar. 25).
54. Hu, Y. et al. Electrostatic-Driven Lamination and Untwisting of β-Sheet Assemblies. ACS Nano 2016, 10 (1), 880-888. doi.org/10.1021/acsnano.5b06011.
55. Isele-Holder, R. et al. Self-Propelled Worm-like Filaments: Spontaneous Spiral Formation, Structure, and Dynamics. Soft Matter 2015, 11 (36), 7181-7190. doi.org/10.1039/C5SM01683E.
56. Zhao, Y.; Hu, X.; Zhang, L.; Wang, D.; King, S. M.; Rogers, S. E.; Wang, J.; Lu, J. R.; Xu, H. Monolayer Wall Nanotubes Self-Assembled from Short Peptide Bolaamphiphiles. Journal of Colloid and Interface Science 2021, 583, 553-562. doi.org/10.1016/j.jcis.2020.09.023.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

What is claimed:

1. A peptide comprising or consisting of SEQ ID NO: 1 or 2, or a sequence having at least 80% identity thereto.

2. The peptide of claim 1, wherein the peptide comprises an amidated C-terminus.

3. A kit comprising:
an L-peptide comprising or consisting of SEQ ID NO: 1 or a sequence having at least 80% identity thereto; and
a D-peptide comprising or consisting of SEQ ID NO: 2 or a sequence having at least 80% identity thereto.

4. The kit of claim 3, wherein each of the L-peptide and the D-peptide comprises an amidated C-terminus.

5. A hydrogel composition comprising an aqueous dispersion medium and at least one peptide selected from:
an L-peptide comprising or consisting of SEQ ID NO: 1 or a sequence having at least 80% identity thereto; and
a D-peptide comprising or consisting of SEQ ID NO: 2 or a sequence having at least 80% identity thereto,
wherein the hydrogel is formed by self-assembly of the at least one peptide in the aqueous dispersion medium.

6. The hydrogel composition of claim 5, wherein the hydrogel comprises only the L-peptide.

7. The hydrogel composition of claim 5, wherein the hydrogel comprises only the D-peptide.

8. The hydrogel composition of claim 5, wherein the hydrogel comprises the L-peptide and the D-peptide.

9. The hydrogel composition of claim 8, wherein the L-peptide and the D-peptide are at an L-peptide: D-peptide (L:D) ratio of about 1:3 to about 3:1.

10. The hydrogel composition of claim 5, wherein the aqueous dispersion medium comprises one or more salts.

11. The hydrogel composition of claim 5, having a pH of about 7 to about 11.

12. The hydrogel composition of claim 3, wherein the at least one peptide is in the hydrogel at an amount of at least about 3% w/v.

13. The hydrogel composition of claim 5, wherein the at least one peptide forms a nanospiral structure.

14. The hydrogel composition of claim 5, wherein the at least one peptide comprises an amidated C terminus.

15. A method of preparing the hydrogel composition of claim 5, the method comprising:
(a) preparing a peptide solution comprising the at least one peptide dissolved in water;
(b) adding a salt buffer to the peptide solution; and
(c) adding a salt to the peptide solution and mixing for at least about 30 minutes and until the peptide solution is at least about pH 7.

16. A pharmaceutical composition comprising the hydrogel composition of claim 5, and an active agent.

17. A method of treating a subject with an active ingredient, the method comprising administering the pharmaceutical composition of claim 16 to the subject.

18. A method of treating damaged tissue in a subject in need thereof, the method comprising:
contacting the damaged tissue with the hydrogel of claim 5.

19. The method of claim 18, wherein the hydrogel is formed on the damaged tissue or the hydrogel is formed in vitro and transferred to the damaged tissue.

20. A method of culturing a cell, the method comprising contacting the cell with the hydrogel composition of claim 5.


SEQUENCE LISTING (8 sequences)

SEQ ID NO: 1 - protein, 5 aa, synthetic construct
KFFFL

SEQ ID NO: 2 - protein, 5 aa, synthetic construct
  SITE 1..5: note = All residues are D-amino acids
KFFFL

SEQ ID NO: 3 - protein, 5 aa, synthetic construct
KYFIL

SEQ ID NO: 4 - protein, 5 aa, synthetic construct
  SITE 1..5: note = All residues are D-amino acids
KYFIL

SEQ ID NO: 5 - protein, 8 aa, synthetic construct
GAGAGAGY

SEQ ID NO: 6 - protein, 6 aa, synthetic construct
EFFFFE

SEQ ID NO: 7 - protein, 6 aa, synthetic construct
KFFFFK

SEQ ID NO: 8 - protein, 4 aa, synthetic construct
FKFE